(19) United States
(12) Patent Application Publication
(10) Pub. No.: US 2012/0035209 A1
(43) Pub. Date: Feb. 9, 2012

(54) Hydroxy Substituted 1H-Imidazopyridines and Methods

(75) Inventors: Joseph F. Dellaria, JR., Saint Paul, MN (US); William H. Moser, Saint Paul, MN (US); Matthew R. Radmer, Saint Paul, MN (US); George W. Griesgraber, Saint Paul, MN (US)

(73) Assignee: PFIZER INC., New York, NY (US)

(21) Appl. No.: 13/277,619

(22) Filed: Oct. 20, 2011

Related U.S. Application Data
(63) Continuation of application No. 12/065,490, filed on Jul. 7, 2008.
(60) Provisional application No. 60/713,704, filed on Sep. 2, 2005.

(51) Int. Cl. A61K 31/437; A61P 35/00; A61P 31/12; C07D 471/04

(52) U.S. Cl. 514/303; 546/118

(57) ABSTRACT

Hydroxy substituted 1H-imidazo[4,5-c]pyridin-4-amines, with a hydroxy substituent at the 2-position, pharmaceutical compositions containing these compounds, methods of making the compounds, intermediates, and methods of use of these compounds as immunomodulators for inducing cytokine biosynthesis in animals and in the treatment of diseases including viral and neoplastic diseases are disclosed.

### CROSS REFERENCE TO RELATED APPLICATIONS

The present invention claims priority to U.S. Provisional Application Ser. No. 60/713,704, filed Sep. 2, 2005, which is incorporated herein by reference.

### BACKGROUND

Certain compounds have been found to be useful as immune response modifiers (IRMs), rendering them useful in the treatment of a variety of disorders. However, there continues to be interest in and a need for compounds that have the ability to modulate the immune response, by induction of cytokine biosynthesis or other means.

### SUMMARY OF THE INVENTION

It has now been found that certain 2-hydroxy-1H-imidazo[4,5-c]pyridin-4-amines modulate cytokine biosynthesis. In one aspect, the present invention provides compounds, which are of the following Formulas I, II, and III:

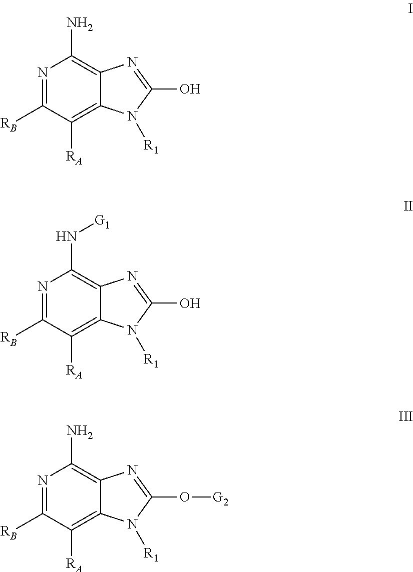

wherein R₁, R_{A}, R_{B}, G₁, and G₂ are as defined below; and pharmaceutically acceptable salts thereof.

The compounds or salts of Formulas I, II, and III are useful as IRMs due to their ability to modulate cytokine biosynthesis (e.g., induce the biosynthesis or production of one or more cytokines) and otherwise modulate the immune response when administered to animals. In some embodiments, compounds or salts of Formula I can be especially useful as immune response modifiers due to their ability to selectively induce interferon (α) (IFN-α), thus providing a benefit over compounds that also induce pro-inflammatory cytokines (e.g. TNF-α) or that induce pro-inflammatory cytokines at higher levels. The ability to modulate cytokine biosynthesis makes the compounds useful in the treatment of a variety of conditions such as viral diseases and neoplastic diseases, that are responsive to such changes in the immune response.

In another aspect, the present invention also provides pharmaceutical compositions containing the compounds of Formulas I, II, and/or III, and methods of inducing cytokine biosynthesis in animal cells, selectively inducing IFN-α in animal cells, treating a viral disease in an animal, and/or treating a neoplastic disease in an animal by administering to the animal one or more compounds of the Formulas I, II, and/or III, and/or pharmaceutically acceptable salts thereof.

In another aspect, the invention provides methods of synthesizing the compounds of Formulas I, II, and III and intermediate compounds useful in the synthesis of these compounds.

As used herein, “a”, “an”, “the”, “at least one”, and “one or more” are used interchangeably.

The terms “comprising” and variations thereof do not have a limiting meaning where these terms appear in the description and claims.

The above summary of the present invention is not intended to describe each disclosed embodiment or every implementation of the present invention. The description that follows more particularly exemplifies illustrative embodiments. Guidance is also provided herein through lists of examples, which can be used in various combinations. In each instance, the recited list serves only as a representative group and should not be interpreted as an exclusive list.

### DETAILED DESCRIPTION OF ILLUSTRATIVE EMBODIMENTS OF THE INVENTION

The present invention provides compounds of the following Formulas I, II, and III:

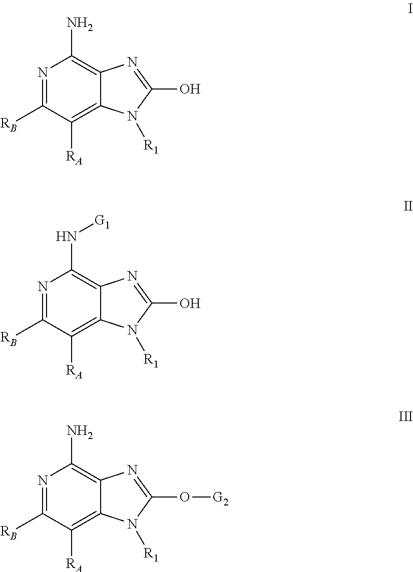

wherein R₁, R_{A}, R_{B}, G₁, and G₂ are as defined below; and pharmaceutically acceptable salts thereof.

In one embodiment, the present invention provides a compound of the following Formula I:

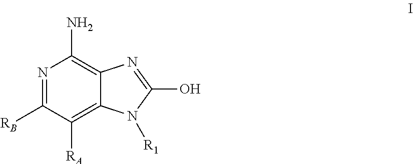

wherein:

R_{A} and R_{B} are each independently selected from the group consisting of:
hydrogen,
halogen,
alkenyl,
amino,
—R₁₁,
—O—Rᵢᵢ,
—S—R₁₁, and
—N(R₉ₐ)(R₁₁);

R₁₁ is selected from the group consisting of alkyl, alkoxyalkylenyl, hydroxyalkylenyl, aryl, arylalkylenyl, heteroaryl, heteroarylalkylenyl, heterocyclyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, heterocyclyl, and heterocyclylalkylenyl, oxo;

R₉ₐ is selected from the group consisting of hydrogen and C₁₋₄ alkyl;

R₁ is selected from the group consisting of:
—R₄,
—X—Y—R₄,
—X—Y—X—Y—R₄,
—X—R₅,
—N(R₁′)-Q-R₄,
—N(R₁′)—X₁—Y₁—R₄, and
—N(R₁′)—X₁—R₅ₐ;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—C(R₆)—O—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

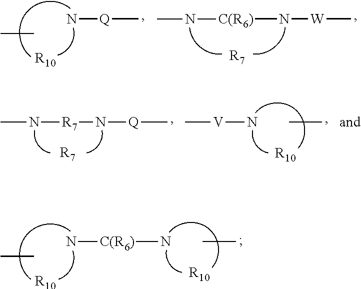

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

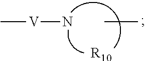

R₁′ is selected from the group consisting of hydrogen, C₁₋₂₀ alkyl, hydroxy-C₂₋₂₀ alkylenyl, and alkoxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl; alkoxy; hydroxyalkyl; haloalkyl; haloalkoxy; halogen; nitro; hydroxy; mercapto; cyano; aryl; aryloxy; arylalkyleneoxy; heteroaryl; heteroaryloxy; heteroarylalkyleneoxy; heterocyclyl; amino; alkylamino; dialkylamino; (dialkylamino)alkyleneoxy; and, in the case of alkyl, alkenyl, alkynyl, and heterocyclyl, oxo;

R₅ is selected from the group consisting of:

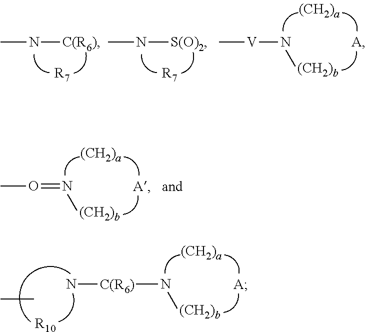

R₅ₐ is selected from the group consisting of:

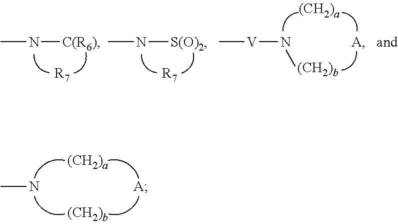

R₆ is selected from the group consisting of ═O and ═S;

R₇ is C₂₋₇ alkylene;

R₈ is selected from the group consisting of hydrogen, C₁₋₁₀ alkyl, C₂₋₁₀ alkenyl, hydroxy-C₁₋₁₀ alkylenyl, C₁₋₁₀ alkoxy-C₁₋₁₀ alkylenyl, aryl-C₁₋₁₀ alkylenyl, and heteroaryl-C₁₋₁₀ alkylenyl;

R₉ is selected from the group consisting of hydrogen and alkyl;

R₁₀ is C₃₋₈ alkylene;

A is selected from the group consisting of —CH₂—, —O—, —C(O)—, —S(O)₀₋₂—, and —N(-Q-R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7; or a pharmaceutically acceptable salt thereof.

In one embodiment, the present invention provides a compound of the following Formula II, which is a prodrug:

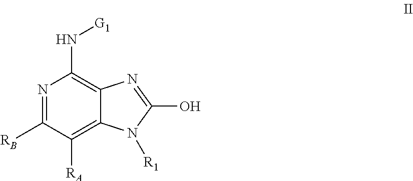

wherein:

G₁ is selected from the group consisting of:
—C(O)—R′,
α-aminoacyl,
α-aminoacyl-α-aminoacyl,
—C(O)—O—R′,
—C(O)—N(R″)R′,
—C(═NY')—R′,
—CH(OH)—C(O)—OY′,
—CH(OC₁₋₄ alkyl)Y₀,
—CH₂Y₂, and
—CH(CH₃)Y₂;

R′ and R″ are independently selected from the group consisting of C₁₋₁₀ alkyl, C₃₋₇ cycloalkyl, phenyl, and benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, aryl-C₁₋₄ alkylenyl, heteroaryl-C₁₋₄alkylenyl, halo-C₁₋₄ alkylenyl, halo-C₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen;

α-aminoacyl is an α-aminoacyl group derived from an amino acid selected from the group consisting of racemic, D-, and L-amino acids;

Y′ is selected from the group consisting of hydrogen, C₁₋₆ alkyl, and benzyl;

Y₀ is selected from the group consisting of C₁₋₆ alkyl, carboxy-C₁₋₆ alkylenyl, amino-C₁₋₄ alkylenyl, mono-N—C₁₋₆alkylamino-C₁₋₄ alkylenyl, and di-N,N—C₁₋₆ alkylamino-C₁₋₄ alkylenyl;

Y₂ is selected from the group consisting of mono-N—C₁₋₆alkylamino, di-N,N—C₁₋₆ alkylamino, morpholin-4-yl, piperidin-1-yl, pyrrolidin-1-yl, and 4-C₁₋₄ alkylpiperazin-1-yl;

R_{A} and R_{B} are each independently selected from the group consisting of:
hydrogen,
halogen,
alkenyl,
amino,
—R₁₁,
—O—R₁₁,
—S—R₁₁, and
—N(R₉ₐ)(R₁₁);

R₁₁ is selected from the group consisting of alkyl, alkoxyalkylenyl, hydroxyalkylenyl, aryl, arylalkylenyl, heteroaryl, heteroarylalkylenyl, heterocyclyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, heterocyclyl, and heterocyclylalkylenyl, oxo;

R₉ₐ is selected from the group consisting of hydrogen and C₁₋₄ alkyl;

R₁ is selected from the group consisting of:
—R₄,
X—R₄,
—X—Y—R₄,
—X—Y—X—Y—R₄,
—X—R₅,
—N(R₁′)-Q-R₄,

—N(R₁′)—X₁—Y₁—R₄, and
—N(R₁′)—X₁—R₅ₐ;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—C(R₆)—O—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,
—O—N(R₈)-Q-,
—O—N═C(R₄)—,
—C(═N—O—R₈)—,
—CH(—N(—O—R₈)-Q-R₄)—,

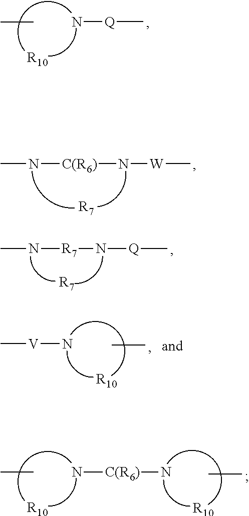

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

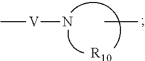

R₁′ is selected from the group consisting of hydrogen, C₁₋₂₀ alkyl, hydroxy-C₂₋₂₀ alkylenyl, and alkoxy-C₂₋₂₀ alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl; alkoxy; hydroxyalkyl; haloalkyl; haloalkoxy; halogen; nitro; hydroxy; mercapto; cyano; aryl; aryloxy; arylalkyleneoxy; heteroaryl; heteroaryloxy; heteroarylalkyleneoxy; heterocyclyl; amino; alkylamino; dialkylamino; (dialkylamino)alkyleneoxy; and, in the case of alkyl, alkenyl, alkynyl, and heterocyclyl, oxo;

R₅ is selected from the group consisting of:

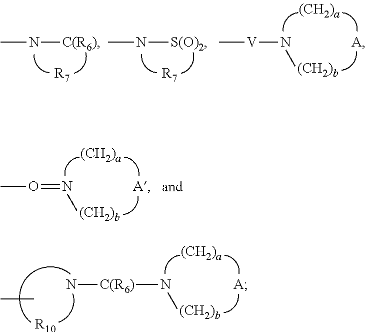

R₅ₐ is selected from the group consisting of:

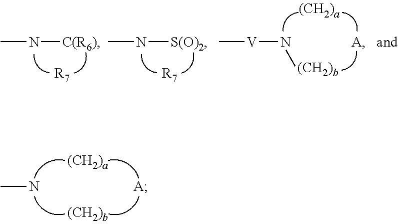

R₆ is selected from the group consisting of ═O and ═S;

R₇ is C₂₋₇ alkylene;

R₃ is selected from the group consisting of hydrogen, C₁₋₁₀ alkyl, C₂₋₁₀ alkenyl, hydroxy-C₁₋₁₀ alkylenyl, C₁₋₁₀ alkoxy-C₁₋₁₀ alkylenyl, aryl-C₁₋₁₀ alkylenyl, and heteroaryl-C₁₋₁₀ alkylenyl;

R₉ is selected from the group consisting of hydrogen and alkyl;

R₁₀ is C₃₋₈ alkylene;

A is selected from the group consisting of —CH₂—, —O—, —C(O)—, —S(O)₀₋₂—, and —N(-Q-R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7; or a pharmaceutically acceptable salt thereof.

In another embodiment, the present invention provides a compound of the following Formula III:

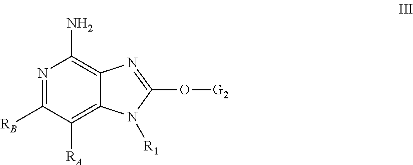

wherein:

G₂ is selected from the group consisting of:
—X₂—C(O)—R′,
α-aminoacyl,
α-aminoacyl-α-aminoacyl,
—X₂—C(O)—O—R′,
—C(O)—N(R″)R′, and
—S(O)₂—R′;

X₂ is selected from the group consisting of a bond; —CH₂—O—; —CH(CH₃)—O—; —C(CH₃)₂—O—; and, in the case of —X₂—C(O)—O—R′, —CH₂—NH—;

R′ and R″ are independently selected from the group consisting of C₁₋₁₀ alkyl, C₃₋₇ cycloalkyl, phenyl, and benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, aryl-C₁₋₄ alkylenyl, heteroaryl-C₁₋₄ alkylenyl, halo-C₁₋₄ alkylenyl, halo-C₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen;

α-aminoacyl is an α-aminoacyl group derived from an amino acid selected from the group consisting of racemic, D-, and L-amino acids;

R_{A} and R_{B} are each independently selected from the group consisting of:
hydrogen,
halogen,
alkenyl,
amino,
—R₁₁,
—O—R₁₁,
—S—R₁₁, and
—N(R₉ₐ)(R₁₁);

R₁₁ is selected from the group consisting of alkyl, alkoxyalkylenyl, hydroxyalkylenyl, aryl, arylalkylenyl, heteroaryl, heteroarylalkylenyl, heterocyclyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, heterocyclyl, and heterocyclylalkylenyl, oxo;

R₉ₐ is selected from the group consisting of hydrogen and C₁₋₄ alkyl;

R₁ is selected from the group consisting of:
—R₄,
—X—R₄,
—X—Y—R₄,
—X—Y—X—Y—R₄,
—X—R₅,
—N(R₁′)-Q-R₄,
—N(R₁′)—X₁—Y₁—R₄, and
—N(R₁′)—X₁—R₅ₐ;

X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups;

X₁ is C₂₋₂₀ alkylene;

Y is selected from the group consisting of:
—O—,
—S(O)₀₋₂—,
—S(O)₂—N(R₈)—,
—C(R₆)—,
—C(R₆)—O—,
—O—C(R₆)—,
—O—C(O)—O—,
—N(R₈)-Q-,
—C(R₆)—N(R₈)—,
—O—C(R₆)—N(R₈)—,
—C(R₆)—N(OR₉)—,
—O—N(R₈)-Q-,
—O—N═C(R₄)—,
—C(═N—O—R₈)—,

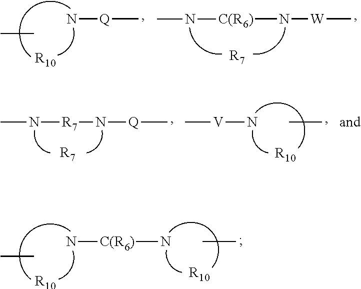

Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

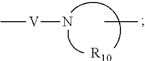

R₁′ is selected from the group consisting of hydrogen, C₁₋₂₀ alkyl, hydroxy-C₂₋₂₀alkylenyl, and alkoxy-C₂₋₂₀alkylenyl;

R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl; alkoxy; hydroxyalkyl; haloalkyl; haloalkoxy; halogen; nitro; hydroxy; mercapto; cyano; aryl; aryloxy; arylalkyleneoxy; heteroaryl; heteroaryloxy; heteroarylalkyleneoxy; heterocyclyl; amino; alkylamino; dialkylamino; (dialkylamino)alkyleneoxy; and, in the case of alkyl, alkenyl, alkynyl, and heterocyclyl, oxo;

R₅ is selected from the group consisting of:

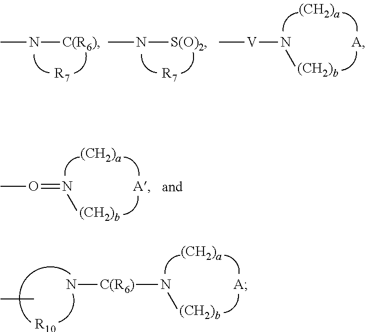

R₅ₐ is selected from the group consisting of:

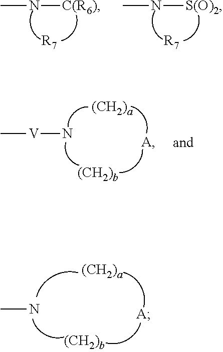

R₆ is selected from the group consisting of ═O and ═S;

R₇ is C₂₋₇ alkylene;

R₈ is selected from the group consisting of hydrogen, C₁₋₁₀ alkyl, C₂₋₁₀ alkenyl, hydroxy-C₁₋₁₀alkylenyl, C₁₋₁₀alkoxy-C₁₋₁₀alkylenyl, aryl-C₁₋₁₀alkylenyl, and heteroaryl-C₁₋₁₀alkylenyl;

R₉ is selected from the group consisting of hydrogen and alkyl;

R₁₀ is C₃₋₈ alkylene;

A is selected from the group consisting of —CH₂—, —O—, —C(O)—, —S(O)₀₋₂—, and —N(-Q-R₄)—;

A′ is selected from the group consisting of —O—, —S(O)₀₋₂—, —N(-Q-R₄)—, and —CH₂—;

Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—;

V is selected from the group consisting of —C(R₆)—, —O—C(R₆)—, —N(R₈)—C(R₆)—, and —S(O)₂—;

W is selected from the group consisting of a bond, —C(O)—, and —S(O)₂—; and

a and b are independently integers from 1 to 6 with the proviso that a+b is ≦7; or a pharmaceutically acceptable salt thereof.

In some embodiments, compounds of Formula III are prodrugs.

For any of the compounds presented herein, each one of the following variables (e.g., R₁, R_{A}, G₁, G₂, R₄, R₁₁, X, X₁, Y, Y₁, A, Q, and so on) in any of its embodiments can be combined with any one or more of the other variables in any of their embodiments and associated with any one of the formulas described herein, as would be understood by one of skill in the art. Each of the resulting combinations of variables is an embodiment of the present invention.

For certain embodiments, e.g., of Formula II, G₁ is selected from the group consisting of —C(O)—W, α-aminoacyl, α-aminoacyl-α-aminoacyl, —C(O)—O—R′, —C(O)—N(R″)R′, —C(═NY′)—R′, —CH(OH)—C(O)—OY′, —CH(OC₁₋₄alkyl)Y₀, —CH₂Y₂, and —CH(CH₃)Y₂. For certain of these embodiments, R′ and R″ are independently selected from the group consisting of C₁₋₁₀ alkyl, C₃₋₇cycloalkyl, phenyl, and benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, aryl-C₁₋₄ alkylenyl, heteroaryl-C₁₋₄ alkylenyl, halo-C₁₋₄ alkylenyl, halo-C₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen;

α-aminoacyl is an α-aminoacyl group derived from an amino acid selected from the group consisting of racemic, D-, and L-amino acids;

Y′ is selected from the group consisting of hydrogen, C₁₋₆ alkyl, and benzyl;

Yₒ is selected from the group consisting of C₁₋₆ alkyl, carboxy-C₁₋₆alkylenyl, amino-C₁₋₄ alkylenyl, mono-N—C₁₋₆alkylamino-C₁₋₄alkylenyl, and di-N,N—C₁₋₆alkylamino-C₁₋₄alkylenyl; and

Y₂ is selected from the group consisting of mono-N—C₁₋₆alkylamino, di-N,N—C₁₋₆alkylamino, morpholin-4-yl, piperidin-1-yl, pyrrolidin-1-yl, and 4-C₁₋₄ alkylpiperazin-1-yl.

For certain embodiments, including any one of the above embodiments of Formula II, G₁ is selected from the group consisting of —C(O)—R′, α-aminoacyl, and —C(O)—O—R′.

For certain embodiments, including any one of the above embodiments of Formula II, G₁ is selected from the group consisting of —C(O)—R′, α-amino-C₂₋₁₁ acyl, and —C(O)—O—R′. α-Amino-C₂₋₁₁ acyl includes α-amino acids containing a total of at least 2 carbon atoms and a total of up to 11 carbon atoms, and may also include one or more heteroatoms selected from the group consisting of O, S, and N.

For certain embodiments, e.g., of Formula III, G₂ is selected from the group consisting of —X₂—C(O)—R′, α-aminoacyl, α-aminoacyl-α-aminoacyl, —X₂—C(O)—O—R′, —C(O)—N(R″)R′, and —S(O)₂—R′. For certain of these embodiments, X₂ is selected from the group consisting of a bond; —CH₂—O—; —CH(CH₃)—O—; —C(CH₃)₂—O—; and, in the case of —X₂—C(O)—O—R′, —CH₂—NH—;

R′ and R″ are independently selected from the group consisting of C₁₋₁₀ alkyl, C₃₋₇ cycloalkyl, phenyl, and benzyl, each of which may be unsubstituted or substituted by one or more substitutents independently selected from the group consisting of halogen, hydroxy, nitro, cyano, carboxy, C₁₋₆ alkyl, C₁₋₄ alkoxy, aryl, heteroaryl, aryl-C₁₋₄ alkylenyl, heteroaryl-C₁₋₄ alkylenyl, halo-C₁ alkylenyl, halo-C₁₋₄ alkoxy, —O—C(O)—CH₃, —C(O)—O—CH₃, —C(O)—NH₂, —O—CH₂—C(O)—NH₂, —NH₂, and —S(O)₂—NH₂, with the proviso that R″ can also be hydrogen; and

α-aminoacyl is an α-aminoacyl group derived from an amino acid selected from the group consisting of racemic, D-, and L-amino acids.

For certain embodiments, including any one of the above embodiments which include an α-aminoacyl group, α-aminoacyl is an α-aminoacyl group derived from a naturally occurring amino acid selected from the group consisting of racemic, D-, and L-amino acids.

For certain embodiments, including any one of the above embodiments which include an α-aminoacyl group, α-aminoacyl is an α-aminoacyl group derived from an amino acid found in proteins, wherein the amino acid is selected from the group consisting of racemic, D-, and L-amino acids.

For certain embodiments, including any one of the above embodiments of Formula III, G₂ is selected from the group consisting of α-amino-C₂₋₅ alkanoyl, C₂₋₆ alkanoyl, C₁₋₆ alkoxycarbonyl, and C₁₋₆ alkylcarbamoyl.

For certain embodiments, the hydrogen atom of the 2-hydroxy substituent of Formula II is replaced by G₂, wherein G₂ is defined as in any one of the above embodiments containing G₂.

For certain embodiments, including any one of the above embodiments of Formula I, II, or III, R_{A} and R_{B} are each independently selected from the group consisting of: hydrogen, halogen, alkenyl, amino, —R₁₁, —O—R₁₁, —S—R₁₁, and —N(R₉ₐ)(R₁₁).

For certain embodiments, when R_{A} and R_{B} or either R_{A} or R_{B} is —R₁₁, R₁₁ is selected from the group consisting of alkyl, alkoxyalkylenyl, hydroxyalkylenyl, arylalkylenyl, heteroarylalkylenyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, heterocyclyl, and heterocyclylalkylenyl, oxo.

For certain embodiments, including any one of the above embodiments of Formula I, II, or III, R_{A} and R_{B} are independently selected from the group consisting of hydrogen, —R₁₁, —O—R₁₁, and —NHR₁₁, wherein R₁₁ is alkyl, alkoxyalkylenyl, or hydroxyalkylenyl. For certain of these embodiments, R_{A} and R_{B} are independently selected from the group consisting of hydrogen, C₁₋₅ alkyl, —O—C₁₋₄ alkyl, C₁₋₄ alkyl-O—C₁₋₄ alkylenyl, and —NH—C₁₋₄ alkyl. For certain of these embodiments, R_{A} and R_{B} are independently selected from the group consisting of hydrogen, C₁₋₅ alkyl, —O—C₁₋₄ alkyl, and —NH—C₁₋₄ alkyl. For certain of these embodiments, R_{A} is selected from the group consisting of hydrogen and C₁₋₅ alkyl, and R_{B} is selected from the group consisting of C₁₋₅ alkyl, —O—C₁₋₄ alkyl, and —NH—C₁₋₄ alkyl. For certain of these embodiments, except where R_{A} and R_{B} cannot be alkyl, R_{A} and R_{B} are independently hydrogen or alkyl. For certain of these embodiments, R_{A} is hydrogen or methyl. For certain of these embodiments, R_{A} is hydrogen. For certain of these embodiments, R_{B} is C₁₋₅ alkyl. For certain of these embodiments, except where R_{A} is hydrogen, R_{A} and R_{B} are each methyl.

For certain embodiments, including any one of the above embodiments of Formula I, II, or III, R₁ is selected from the group consisting of —R₄, —X—R₄, —X—Y—R₄, —X—Y—X—Y—R₄, and —X—R₅.

For certain embodiments, including any one of the above embodiments of Formula I, II, or III, R₁ is —R₄ or —X—R₄.

For certain embodiments, including any one of the above embodiments of Formula I, II, or III, R₁ is selected from the group consisting of aryl-C₁₋₄alkylenyl and heteroaryl-C₁₋₄ alkylenyl, wherein the aryl or heteroaryl group is unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and (dialkylamino)alkyleneoxy. For certain of these embodiments, R₁ is benzyl, which is unsubstituted or substituted by one or more substituents independently selected from the group consisting of alkyl, alkoxy, haloalkyl, haloalkoxy, and halogen. For certain of these embodiments, R₁ is benzyl or 4-fluorobenzyl.

For certain of these embodiments, including any one of the above embodiments of Formula I, II, or III where R₁ is or includes —X—R₄, —X— is

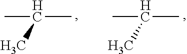

—CH₂—, —(CH₂)₂—, —CH(CH₃)—, —(CH₂)₃—, or —(CH₂)₄—. Alternatively, X is —C₁₋₄alkylene-O—C₁₋₄alkylene-. For certain of these embodiments, X is —(CH₂)₂—O—(CH₂)₃—.

For certain embodiments, including any one of the above embodiments of Formula I, II, or III, where not excluded, R₁ is tetrahydro-2H-pyran-4-ylmethyl.

For certain embodiments, including any one of the above embodiments of Formula I, II, or III, where not excluded, R₁ is pyridin-3-ylmethyl, isoxazol-5-ylmethyl, isoxazol-3-ylmethyl, [3-methylisoxazol-5-yl]methyl, [5-(4-fluorophenypisoxazol-3-yl]methyl, or [3-(4-fluorophenypisoxazol-5-yl]methyl. For certain of these embodiments, R₁ is pyridin-3-ylmethyl, isoxazol-5-ylmethyl, isoxazol-3-ylmethyl, [5-(4-fluorophenypisoxazol-3-yl]methyl, or [3-(4-fluorophenyl)isoxazol-5-yl]methyl.

For certain embodiments, including any one of the above embodiments of Formula I, II, or III, except where R₁ is —R₄ or —X—R₄, R₁ is —X—Y—R₄. For certain of these embodiments, R₁ is —C₂₋₅alkylenyl-S(O)₂—C₁₋₃ alkyl. Alternatively, for certain of these embodiments, R₁ is

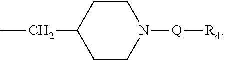

Alternatively, for certain of these embodiments, R₁ is —C₂₋₅ alkylenyl-NH-Q-R₄. For certain of these embodiments where Q is present, Q is —C(O)—, S(O)₂—, or —C(O)—NH—, and R₄ is C₁₋₆alkyl.

For certain embodiments, including any one of the above embodiments of Formula I, II, or III, where not excluded, R₁ is selected from the group consisting of —N(R₁′)-Q-R₄, —N(R₁′)—X₁—Y₁—R₄, and —N(R₁′)—X₁—R₅ₐ.

For certain embodiments, including any one of the above embodiments of Formula I, II, or III, where not excluded, R₁ is —N(R₁′)-Q-R₄. For certain of these embodiments, R₁′ is hydrogen, Q is a bond, and R₄ is aryl, heteroaryl, aryl-C₁₋₃alkylenyl, or heteroaryl-C₁₋₃alkylenyl.

For certain embodiments, R₁′ is selected from the group consisting of hydrogen, C₁₋₂₀ alkyl, hydroxy-C₂₋₂₀alkylenyl, and alkoxy-C₂₋₂₀alkylenyl.

For certain embodiments, R₁′ is hydrogen or methyl.

For certain embodiments, R₁′ is hydrogen.

For certain embodiments, R₄ is selected from the group consisting of hydrogen, alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl wherein the alkyl, alkenyl, alkynyl, aryl, arylalkylenyl, aryloxyalkylenyl, alkylarylenyl, heteroaryl, heteroarylalkylenyl, heteroaryloxyalkylenyl, alkylheteroarylenyl, and heterocyclyl groups can be unsubstituted or substituted by one or more substituents independently selected from the group consisting of alkyl; alkoxy; hydroxyalkyl; haloalkyl; haloalkoxy; halogen; nitro; hydroxy; mercapto; cyano; aryl; aryloxy; arylalkyleneoxy; heteroaryl; heteroaryloxy; heteroarylalkyleneoxy; heterocyclyl; amino; alkylamino; dialkylamino; (dialkylamino)alkyleneoxy; and, in the case of alkyl, alkenyl, alkynyl, and heterocyclyl, oxo.

For certain embodiments, R₄ is selected from the group consisting of aryl-C₁₋₄alkylenyl and heteroaryl-C₁₋₄ alkylenyl, wherein the aryl or heteroaryl group is unsubstituted or substituted by one or more substituents independently selected from the group consisting of alkyl, alkoxy, hydroxyalkyl, haloalkyl, haloalkoxy, halogen, nitro, hydroxy, mercapto, cyano, aryl, aryloxy, arylalkyleneoxy, heteroaryl, heteroaryloxy, heteroarylalkyleneoxy, heterocyclyl, amino, alkylamino, dialkylamino, and (dialkylamino)alkyleneoxy.

For certain embodiments, R₄ is benzyl, which is unsubstituted or substituted by one or more substituents independently selected from the group consisting of alkyl, alkoxy, haloalkyl, haloalkoxy, and halogen.

For certain embodiments, R₄ is benzyl.

For certain embodiments, R₄ is tetrahydro-2H-pyran-4-ylmethyl.

For certain embodiments, R₄ is aryl, heteroaryl, aryl-C₁₋₃ alkylenyl, or heteroaryl-C₁₋₃ alkylenyl.

For certain embodiments, R₄ is isoxazol-3-yl, isoxazol-5-yl, or thiazol-2-yl, each of which is unsubstituted or substituted by methyl or 4-fluorophenyl.

For certain embodiments, R₄ is phenyl.

For certain embodiments, R₄ is C₁₋₆ alkyl.

For certain embodiments, R₄ is C₁₋₃ alkyl.

For certain embodiments, R₅ₐ is selected from the group consisting of:

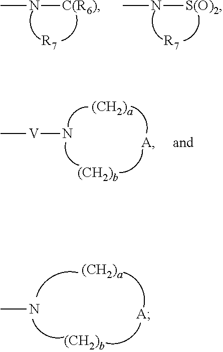

For certain embodiments, R₅ₐ is

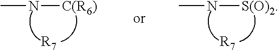

For certain embodiments, R₅ₐ is

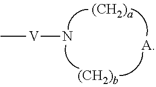

For certain embodiments, R₅ is selected from the group consisting of:

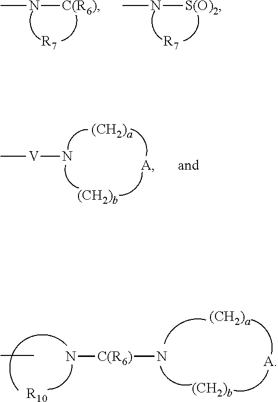

For certain embodiments, R₅ is

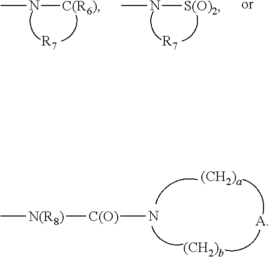

For certain embodiments, R₆ is selected from the group consisting of ═O and ═S.

For certain embodiments, R₆ is ═O.

For certain embodiments, R₇ is C₂₋₇ alkylene.

For certain embodiments, R₇ is C₂₋₄ alkylene.

For certain embodiments, R₇ is ethylene.

For certain embodiments, R₈ is selected from the group consisting of hydrogen, C₁₋₁₀ alkyl, C₂₋₁₀ alkenyl, hydroxy-C₁₋₁₀ alkylenyl, C₁₋₁₀ alkoxy-C₁₋₁₀ alkylenyl, aryl-C₁₋₁₀ alkylenyl, and heteroaryl-C₁₋₁₀ alkylenyl.

For certain embodiments, R₈ is hydrogen or C₁₋₄ alkyl.

For certain embodiments, R₈ is hydrogen.

For certain embodiments, R₉ₐ is selected from the group consisting of hydrogen and C₁₋₄ alkyl.

For certain embodiments, R₉ₐ is hydrogen.

For certain embodiments, R₉ is selected from the group consisting of hydrogen and alkyl.

For certain embodiments, R₁₀ is C₃₋₈ alkylene.

For certain embodiments, R₁₀ is pentylene.

For certain embodiments, R₁₁ is selected from the group consisting of alkyl, alkoxyalkylenyl, hydroxyalkylenyl, aryl, arylalkylenyl, heteroaryl, heteroarylalkylenyl, heterocyclyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, heterocyclyl, and heterocyclylalkylenyl, oxo.

For certain embodiments, R₁₁ is selected from the group consisting of alkyl, alkoxyalkylenyl, hydroxyalkylenyl, arylalkylenyl, heteroarylalkylenyl, and heterocyclylalkylenyl, each of which is unsubstituted or substituted by one or more substituents independently selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and, in the case of alkyl, and heterocyclylalkylenyl, oxo.

For certain embodiments, R₁₁ is alkyl, alkoxyalkylenyl, or hydroxyalkylenyl.

For certain embodiments, R₁₁ is pentyl.

For certain embodiments, A is selected from the group consisting of —CH₂—, —O—, —C(O)—, —S(O)₀₋₂—, and —N(-Q-R₄)—. For certain embodiments, A is —O—.

For certain embodiments, A′ is selected from the group consisting of —O—, —S(O)₀₋₂—, —N(-Q-R₄)—, and —CH₂—.

For certain embodiments, Q is selected from the group consisting of a bond, —C(R₆)—, —C(R₆)—C(R₆)—, —S(O)₂—, —C(R₆)—N(R₈)—W—, —S(O)₂—N(R₈)—, —C(R₆)—O—, —C(R₆)—S—, and —C(R₆)—N(OR₉)—. For certain embodiments, Q is —C(R₆)—N(R₈)—, —C(R₆)—, or —S(O)₂—.

For certain embodiments, Q is —C(O)—N(H)—, —C(O)—, or —S(O)₂—. For certain embodiments, Q is —C(R₆)—N(R₈)—. For certain embodiments, Q is —C(O)—NH—. For certain embodiments, Q is —S(O)₂—. For certain embodiments, Q is —C(R₆)—. For certain embodiments, Q is —C(O)—. For certain embodiments, Q is a bond.

For certain embodiments, V is selected from the group consisting of —C(R₆)—, —O—C(R₆)—, —N(R₈)—C(R₆)—, and —S(O)₂—. For certain embodiments, V is —N(R₈)—C(O)—.

For certain embodiments, W is selected from the group consisting of a bond, —C(O)—, and —S(O)₂—. For certain embodiments, W is a bond.

For certain embodiments, X is selected from the group consisting of alkylene, alkenylene, alkynylene, arylene, heteroarylene, and heterocyclylene wherein the alkylene, alkenylene, and alkynylene groups can be optionally interrupted or terminated by arylene, heteroarylene or heterocyclylene and optionally interrupted by one or more —O— groups.

For certain embodiments, X is C₁₋₄ alkylene.

For certain embodiments, —X— is

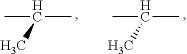

### —CH₂—, —(CH₂)₂—, —CH(CH₃)—, —(CH₂)₃—, or —(CH₂)₄—.

For certain embodiments, X is methylene.

For certain embodiments, X is —C₁₋₄ alkylene-O—C₁₋₄ alkylene-.

For certain of these embodiments, X is —(CH₂)₂—O—(CH₂)₃—.

For certain embodiments, X₁ is C₂₋₂₀ alkylene. For certain embodiments, X₁ is C₂₋₄ alkylene.

For certain embodiments, Y is selected from the group consisting of —O—, —S(O)₀₋₂—, —S(O)₂—N(R₈)—, —C(R₆)—, —C(R₆)—O—, —O—C(R₆)—, —N(R₈)-Q-, —C(R₆)—N(R₈)—, —O—C(R₆)—N(R₈)—, —C(R₆)—N(OR₉)—, —O—N(R₈)-Q-, —O—N═C(R₄)—, —C(═N—O—R₈)—, —CH(—N(—O—R₈)-Q-R₄-,

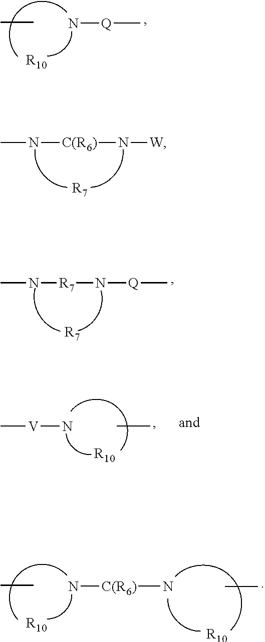

For certain embodiments, Y is —N(R₈)—C(O)—, —N(R₈)—S(O)₂—, —N(R₈)—C(R₆)—N(R₈)—, —N(R₈)—C(R₆)—N(R₈)—C(O)—, —N(R₈)—C(R₆)—O—,

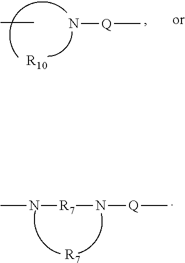

For certain embodiments, Y is —S(O)₂—.

For certain embodiments, Y is —NH-Q-.

For certain embodiments, Y is

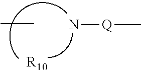

For certain embodiments, Y is

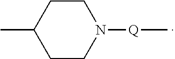

For certain embodiments, Y₁ is selected from the group consisting of —O—, —S(O)₀₋₂—, —S(O)₂—N(R₈)—, —N(R₈)-Q-, —C(R₆)—N(R₈)—, —O—C(R₆)—N(R₈)—, and

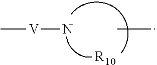

For certain embodiments, a and b are independently integers from 1 to 6 with the proviso that a+b is ≦7. For certain embodiments, a and b are each 2.

For certain embodiments, the present invention provides a pharmaceutical composition comprising a therapeutically effective amount of a compound or salt of any one of the above embodiments of Formulas I, II, and III, and a pharmaceutically acceptable carrier.

For certain embodiments, the present invention provides a method of inducing cytokine biosynthesis in an animal comprising administering an effective amount of a compound or salt of any one of the above embodiments of Formulas I, II, and III, or a pharmaceutical composition comprising an effective amount of any one of the above embodiments of Formulas I, II, and III to the animal. For certain of these embodiments, the cytokine is selected from the group consisting of IFN-α, TNF-α, IL-6, and IL-10. For certain of these embodiments, the cytokine is IFN-α or IFN-α and TNF-α. For certain of these embodiments, the cytokine is IFN-α.

For certain embodiments, the present invention provides a method of selectively inducing the biosynthesis of IFN-α in an animal comprising administering an effective amount of a compound or salt of any one of the above embodiments of Formulas I, II, and III, or a pharmaceutical composition comprising an effective amount of any one of the above embodiments of Formulas I, II, and III to the animal.

For certain embodiments, the present invention provides a method of treating a viral disease in an animal comprising administering a therapeutically effective amount of a compound or salt of any one of the above embodiments of Formulas I, II, and III, or a pharmaceutical composition comprising a therapeutically effective amount of any one of the above embodiments of Formulas I, II, and III to the animal.

For certain embodiments, the present invention provides a method of treating a viral disease in an animal comprising administering a therapeutically effective amount of a compound or salt of any one of the above embodiments of Formulas I, II, and III, or a pharmaceutical composition comprising a therapeutically effective amount of any one of the above embodiments of Formulas I, II, and III the animal; and selectively inducing the biosynthesis of IFN-α in the animal.

For certain embodiments, the present invention provides a method of treating a neoplastic disease in an animal comprising administering a therapeutically effective amount of a compound or salt of any one of the above embodiments of Formulas I, II, and III, or a pharmaceutical composition comprising a therapeutically effective amount of any one of the above embodiments of Formulas I, II, and III to the animal.

For certain embodiments, the present invention provides a method of treating a neoplastic disease in an animal comprising administering a therapeutically effective amount of a compound or salt of any one of the above embodiments of Formulas I, II, and III, or a pharmaceutical composition comprising a therapeutically effective amount of any one of the above embodiments of Formulas I, II, and III to the animal; and selectively inducing the biosynthesis of IFN-α in the animal.

As used herein, the terms “alkyl”, “alkenyl”, “alkynyl” and the prefix “alk-” are inclusive of both straight chain and branched chain groups and of cyclic groups, e.g., cycloalkyl and cycloalkenyl. Unless otherwise specified, these groups contain from 1 to 20 carbon atoms, with alkenyl groups containing from 2 to 20 carbon atoms, and alkynyl groups containing from 2 to 20 carbon atoms. In some embodiments, these groups have a total of up to 10 carbon atoms, up to 8 carbon atoms, up to 6 carbon atoms, or up to 4 carbon atoms. Cyclic groups can be monocyclic or polycyclic and preferably have from 3 to 10 ring carbon atoms. Exemplary cyclic groups include cyclopropyl, cyclopropylmethyl, cyclobutyl, cyclobutylmethyl, cyclopentyl, cyclopentylmethyl, cyclohexyl, cyclohexylmethyl, adamantyl, and substituted and unsubstituted bornyl, norbornyl, and norbornenyl.

Unless otherwise specified, “alkylene”, “-alkylene-”, “alkenylene”, “-alkenylene-”, “alkynylene”, and “-alkynylene-” are the divalent forms of the “alkyl”, “alkenyl”, and “alkynyl” groups defined above. The terms “alkylenyl”, “alkenylenyl”, and “alkynylenyl” are used when “alkylene”, “alkenylene”, and “alkynylene”, respectively, are substituted. For example, an arylalkylenyl group comprises an “alkylene” moiety to which an aryl group is attached.

The term “haloalkyl” is inclusive of alkyl groups that are substituted by one or more halogen atoms, including perfluorinated groups. This is also true of other groups that include the prefix “halo-”. Examples of suitable haloalkyl groups are chloromethyl, trifluoromethyl, and the like.

The term “aryl” as used herein includes carbocyclic aromatic rings or ring systems. Examples of aryl groups include phenyl, naphthyl, biphenyl, fluorenyl and indenyl.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyriinidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The term “heterocyclyl” includes non-aromatic rings or ring systems that contain at least one ring heteroatom (e.g., O, S, N) and includes all of the fully saturated and partially unsaturated derivatives of the above mentioned heteroaryl groups. In some embodiments, the term “heterocyclyl” includes a ring or ring system that contains 2 to 12 carbon atoms, 1 to 3 rings, 1 to 4 heteroatoms, and O, S, and N as the heteroatoms. Exemplary heterocyclyl groups include pyrrolidinyl, tetrahydropyranyl, morpholinyl, thiomorpholinyl, 1,1-dioxothiomorpholinyl, piperidinyl, piperazinyl, thiazolidinyl, imidazolidinyl, isothiazolidinyl, tetrahydropyranyl, quinuclidinyl, homopiperidinyl (azepanyl), 1,4-oxazepanyl, homopiperazinyl (diazepanyl), 1,3-dioxolanyl, aziridinyl, azetidinyl, dihydroisoquinolin-(1H)-yl, octahydroisoquinolin-(1H)-yl, dihydroquinolin-(2H)-yl, octahydroquinolin-(2H)-yl, dihydro-1H-imidazolyl, 3-azabicyclo[3.2.2]non-3-yl, and the like.

The term “heterocyclyl” includes bicyclic and tricyclic heterocyclic ring systems. Such ring systems include fused and/or bridged rings and Spiro rings. Fused rings can include, in addition to a saturated or partially saturated ring, an aromatic ring, for example, a benzene ring. Spiro rings include two rings joined by one Spiro atom and three rings joined by two Spiro atoms.

When “heterocyclyl” contains a nitrogen atom, the point of attachment of the heterocyclyl group may be the nitrogen atom.

The terms “arylene”, “heteroarylene”, and “heterocyclylene” are the divalent forms of the “aryl”, “heteroaryl”, and “heterocyclyl” groups defined above. The terms, “arylenyl”, “heteroarylenyl”, and “heterocyclylenyl” are used when “arylene”, “heteroarylene”, and “heterocyclylene”, respectively, are substituted. For example, an alkylarylenyl group comprises an arylene moiety to which an alkyl group is attached.

When a group (or substituent or variable) is present more than once in any Formula described herein, each group (or substituent or variable) is independently selected, whether explicitly stated or not. For example, for the formula

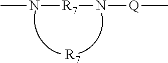

each R₇ group is independently selected. In another example, when more than one Y group is present, each Y group is independently selected. In a further example, when more than one —N(R₈)—C(R₆)—N(R₈)— group is present (e.g., more than one Y group is present, and both contain a —N(R₈)—C(R₆)—N(R₈)— group) each R₈ group is independently selected and each R₆ group is independently selected.

The invention is inclusive of the compounds described herein (including intermediates) in any of their pharmaceutically acceptable forms, including isomers (e.g., diastereomers and enantiomers), salts, solvates, polymorphs, prodrugs, and the like. In particular, if a compound is optically active, the invention specifically includes each of the compound's enantiomers as well as racemic and scalemic mixtures of the enantiomers. It should be understood that the term “compound” includes any or all of such forms, whether explicitly stated or not (although at times, “salts” are explicitly stated).

The term “prodrug” means a compound that can be transformed in vivo to yield an immune response modifying compound, including any of the salt, solvated, polymorphic, or isomeric forms described above. The prodrug, itself, may be an immune response modifying compound, including any of the salt, solvated, polymorphic, or isomeric forms described above. The transformation may occur by various mechanisms, such as through a chemical (e.g., solvolysis or hydrolysis, for example, in the blood) or enzymatic biotransformation. A discussion of the use of prodrugs is provided by T. Higuchi and W. Stella, “Pro-drugs as Novel Delivery Systems,” Vol. 14 of the A. C. S. Symposium Series, and in Bioreversible Carriers in Drug Design, ed. Edward B. Roche, American Pharmaceutical Association and Pergamon Press, 1987.

### Preparation of the Compounds

Compounds of the invention may be synthesized by synthetic routes that include processes analogous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g. prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

In the preparation of compounds of the invention it may sometimes be necessary to protect a particular functionality while reacting other functional groups on an intermediate. The need for such protection will vary depending on the nature of the particular functional group and the conditions of the reaction step. Suitable amino protecting groups include acetyl, trifluoroacetyl, tert-butoxycarbonyl (Boc), benzyloxycarbonyl, and 9-fluorenylmethoxycarbonyl (Fmoc). Suitable hydroxy protecting groups include acetyl and silyl groups such as the tert-butyl dimethylsilyl group. For a general description of protecting groups and their use, see T. W. Greene and P. G. M. Wuts, Protective Groups in Organic Synthesis, John Wiley & Sons, New York, USA, 1991.

Conventional methods and techniques of separation and purification can be used to isolate compounds of the invention or pharmaceutically acceptable salts thereof, as well as various intermediates related thereto. Such techniques may include, for example, all types of chromatography (high performance liquid chromatography (HPLC), column chromatography using common absorbents such as silica gel, and thin layer chromatography, recrystallization, and differential (i.e., liquid-liquid) extraction techniques.

Compounds of the invention can be prepared according to Reaction Scheme I, wherein R_{A}, R_{B}, G₁, G₂, and R₁ are as defined above, and Bn is benzyl, p-methoxybenzyl, p-methylbenzyl, or 2-furanylmethyl. In step (1) of Reaction Scheme I, a 2,4-dichloro-3-nitropyridine of Formula V is reacted with an amine of Formula R₁—NH₂. The reaction can be conveniently carried out by adding the amine to a solution of a compound of Formula V in the presence of a base such as triethylamine. The reaction is carried out in a suitable solvent, such as dichloromethane, chloroform, or N,N-dimethylformamide (DMF) and may be carried out at room temperature, a sub-ambient temperature such as 0° C., or an elevated temperature such as the reflux temperature of the solvent. Many 2,4-dichloro-3-nitropyridines of Formula V are known or can be prepared by known methods; see, for example, U.S. Pat. No. 6,525,064 (Dellaria et al.). For example, they are readily prepared by chlorinating 4-hydroxy-3-nitro-2(1H)-pyridones with a chlorinating agent such as phosphorus(III) oxychloride. Many 4-hydroxy-3-nitro-2(1H)-pyridones are known or can be prepared by known methods; see, for, example, U.S. Pat. No. 5,446,153 (Lindstrom et al.) and the references cited therein. Other 2,4-dichloro-3-nitropyridines of Formula V can be prepared according to methods described in Reaction Scheme II.

Numerous amines of Formula R₁—NH₂ are commercially available; others can be prepared by known methods. For example, a variety of substituted and unsubstituted alkyl and arylalkylenyl amines, isomeric (aminomethyl)pyridines, and alkyl, aryl, or arylalkylenyl hydrazines or hydrazine salts are commercially available. In certain preferred embodiments, R₁ is a (5-substituted-isoxazol-3-yl)methyl group. (5-Substituted-isoxazol-3-yl)methylamines can be prepared by the following four-step method. In part (i), a protected amino-aldehyde of formula (PG)₂-X—CH═O, wherein PG is a nitrogen protecting group and X is as defined above, is converted to an aldoxime of formula (PG)₂-X—CH═N—OH using conventional methods. For example, an aldehyde can be combined with hydroxylamine hydrochloride in the presence of base such as triethylamine in a suitable solvent such as dichloromethane. The reaction can be run at room temperature. Protected amino-aldehydes can be prepared using conventional methods. For example, phthalimidoacetaldehyde diethyl acetal is a commercially available compound that can be treated with acid to provide an aldehyde of formula (PG)₂-X—CH═O. In part (ii), an aldoxime of formula (PG)₂-X—CH═N—OH is converted to an α-chloroaldoxime of formula (PG)₂-X—C(Cl)═N—OH by treatment with N-chlorosuccinimide in a suitable solvent such as DMF. The reaction may be carried out initially below room temperature, at 0° C. for example, and then heated at an elevated temperature in the range of 40° C. to 50° C. The α-chloroaldoxime of formula (PG)₂-X—C(Cl)═N—OH can optionally be isolated before it is converted in part (iii) to a protected (5-substituted-isoxazol-3-yl)methylamine by treatment with a base such as triethylamine to generate a nitrile oxide in the presence of an alkyne in a suitable solvent such as dichloromethane at room temperature. The nitrile oxide and alkyne undergo a [3+2]cycloaddition reaction to provide a protected (5-substituted-isoxazol-3-yl)methylamine, which is then deprotected in part (iv) using conventional methods. When a phthalimide protecting group is used, the deprotection can be carried out by combining the phthalimide-protected (5-substituted-isoxazol-3-yl)methylamine with hydrazine or hydrazine hydrate in a suitable solvent such as ethanol or solvent mixture such as ethanol/THF. The deprotection reaction can be carried out at room temperature or at an elevated temperature such as the reflux temperature of the solvent.

Some amines of the Formula H₂N—R₁ can be made according to the following methods. For some embodiments, R₁ is a 1-hydroxycycloalkylmethyl group, a (4-hydroxytetrahydro-2H-pyran-4-yl)methyl group, or a group derived from a [1-(tert-butoxycarbonyl)-4-hydroxypiperidin-4-yl]methyl group. The corresponding amines of Formula H₂N—R₁ can be prepared by combining a cyclic ketone, such as cyclopentanone, cyclobutanone, tetrahydro-4H-pyran-4-one, and tert-butyl 4-oxo-1-piperidinecarboxylate, with excess nitromethane in a suitable solvent such as ethanol or methanol in the presence of a catalytic amount of base such as sodium ethoxide or sodium hydroxide and reducing the resultant nitromethyl-substituted compound using conventional heterogeneous hydrogenation conditions. The hydrogenation is typically carried out in the presence of a catalyst such as palladium hydroxide on carbon, palladium on carbon, or Raney nickel in a suitable solvent such as ethanol. Both the reaction with nitromethane and the reduction can be carried out at room temperature. A wide variety of cyclic ketones can be obtained from commercial sources; others can be synthesized using known synthetic methods.

In step (2) of Reaction Scheme I, the chloro group in a pyridine of Formula VI is displaced by an amine of Formula HN(Bn)₂ to provide a pyridine of Formula VII. The displacement is conveniently carried out by combining an amine of Formula HN(Bn)₂ and a compound of Formula VI in a suitable solvent such as toluene or xylenes in the presence of a base such as triethylamine and heating at an elevated temperature such as the reflux temperature of the solvent.

In step (3) of Reaction Scheme I, a compound of Formula VII is reduced to provide a pyridine-2,3,4-triamine of Formula VIII. The reduction can be carried out using nickel boride, prepared in situ from sodium borohydride and nickel(II) chloride. The reduction is conveniently carried out by adding a solution of a pyridine of Formula VII in a suitable solvent or solvent mixture such as dichloromethane/methanol to a mixture of excess sodium borohydride and catalytic or stoichiometric nickel(II) chloride in methanol. The reaction can be carried out at room temperature.

In step (4) of Reaction Scheme I, a pyridine-2,3,4-triamine of Formula VIII is cyclized to provide a 1H-imidazo[4,5-e]pyridin-2-ol of Formula IX. The cyclization can be conveniently carried out by heating a pyridine-2,3,4-triamine of Formula VIII with carbonyl diimidazole in a suitable solvent such as tetrahydrofuran (THF), tert-butyl methyl ether, dichloromethane, or DMF. The reaction may be carried out at room temperature or, preferably, at an elevated temperature such as the reflux temperature of the solvent.

In step (5) of Reaction Scheme I, the protecting groups are removed from the 4-amine of a 1H-imidazo[4,5-c]pyridin-2-ol of Formula IX to provide a 1H-imidazo[4,5-c]pyridin-2-ol of Formula I. For certain embodiments, the deprotection can be conveniently carried out on a Parr apparatus under hydrogenolysis conditions using a suitable heterogeneous catalyst such as palladium on carbon in a solvent such as ethanol. Alternatively, when Bn is p-methoxybenzyl, step (5) may carried out by combining trifluoroacetic acid and a compound of Formula IX and stirring at room temperature or heating at an elevated temperature such as 50° C. to 70° C.

Certain amines of Formula R₁—NH₂ provide a compound of Formula VI that contain a functional group or protected functional group that can be transformed in a subsequent step to provide compounds of Formula I with a variety of different R₁ groups. For example, protected diamines of Formula Boc-N(R₈)—X—NH₂,

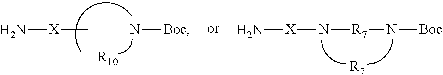

are commercially available or can be prepared by known methods; see, for example, U.S. Pat. No. 6,797,718 (Dellaria et al.) and Carceller, E. et al., J. Med. Chem., 39, pp. 487-493 (1996). The Boc-protected amino group can be subjected to the reaction conditions of steps (2) through (4) of Reaction Scheme I. The Boc-protecting group may be removed in step (5) if the acidic conditions are used, or it can be removed by conventional methods after step (5). The resulting compound of Formula I having an —X—N(R₈)H,

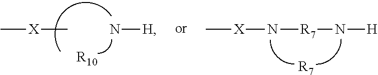

group at the R₁ position can be converted to a compound having an —X—N(R₈)-Q-R₄,

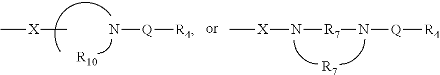

group using known methods; see, for example, Dellaria et al. in U.S. Pat. Nos. 6,525,064, 6,545,016, 6,545,017, and 6,7979,718. In other examples, amino esters of Formula H₂N—X—C(O)—O-alkyl or hydrochloride salts thereof can be used in step (1), and the resulting compound of Formula VI can be converted in subsequent steps to a compound of Formula I having an —X—C(R₆)—R₄, —X—C(R₆)—N(OR₉)—R₄, or —X—C(═N—O—R₈)—R₄ group at the R₁ position using the methods described in International Publication Nos. WO2005/051317 (Krepski et al.) and WO2005/051324 (Krepski et al.). Compounds of Formula VI prepared from amino esters can also be used to prepare compounds of Formula I having an —X—C(R₆)—N(R₈)—R₄ group at the R₁ position using conventional acyl transfer reaction conditions. Amino alcohols of Formula H₂N—X—OH can be used to prepare compounds of Formula VI, which can be converted in subsequent steps to a compound of Formula I having an —X—S(O)₀₋₂—R₄, —X—S(O)₀₋₂—N(R₈)—R₄, —X—O—N(R₈)-Q-R₄, —X—O—N═C(R₄)—R₄, —X—CH(—N(—O—R₈)-Q-R₄)—R₄ group using methods described in U.S. Pat. No. 6,797,718 (Dellaria et al.) and International Publication Nos. WO2005/066169 (Bonk and Dellaria), WO2005/018551 (Kshirsagar et al.), WO2005/018556 (Kshirsagar et al.), and WO2005/051324 (Krepski et al.), respectively.

The amine used in step (1) may be tert-butyl carbazate, and the resulting tert-butyl 2-(2-chloro-3-nitropyridin-4-yl)hydrazinecarboxylate can be subjected to the conditions of steps (2) to (4). The compound of Formula IX wherein R₁ is a Boc-protected amino group can be deprotected to provide a 1-amino compound or a salt (for example, hydrochloride salt) thereof. The deprotection can be carried out by heating at reflux a solution of a compound of Formula IX in ethanolic hydrogen chloride. The resulting compound of Formula IX wherein R₁ is an amino group can treated with a ketone, aldehyde, or corresponding ketal or acetal thereof, under acidic conditions. For example, a ketone can be added to a solution of the hydrochloride salt of a compound of Formula IX in which R₁ is an amino group in a suitable solvent such as isopropanol or acetonitrile in the presence of an acid such as pyridinium p-toluene sulfonate or acetic acid, or an acid resin, for example, DOWEX W50-X1 acid resin. The reaction can be performed at an elevated temperature. The resulting imine can be reduced to provide a compound of Formula IX in which R₁ is —N(R₁′)-Q-R₄, wherein Q is a bond The reduction can be carried out at room temperature with sodium borohydride in a suitable solvent, for example, methanol. The deprotection shown in step (5) can then be carried out to provide a compound of Formula I. A test-butyl 2-(2-chloro-3-nitropyridin-4-yl)hydrazinecarboxylate of Formula VI can also be manipulated in subsequent steps using the methods described in International Publication No. WO2006/026760 (Stoermer et al.) to provide other compounds of Formula I, wherein R₁ is —N(R₁′)-Q-R₄, —N(R₁′)—X₁—Y₁—R₄, or —N(R₁′)—X₁—R_{5b}.

In certain preferred embodiments, R₁ is a (3-substituted-isoxazol-5-yl)methyl group. This group can be prepared by using propargyl amine as the amine of Formula R₁—NH₂ in step (1) of Reaction Scheme Ito provide a compound of Formula VI wherein R₁ is —CH₂—C≡CH. Prior to step (5) of Reaction Scheme I, the alkyne group at the R₁ position undergoes a cycloaddition reaction with a nitrile oxide formed from an α-chloroaldoxime to provide an isoxazole-substituted 1H-imidazo[4,5-c]pyridin-2-ol of Formula I. α-Chloroaldoximes can be prepared by treating an aldoxime with N-chlorosuccinimide in a suitable solvent such as DMF. The reaction may be carried out initially below room temperature, at 0° C. for example, and then heated at an elevated temperature in the range of 40° C. to 50° C. Aldoximes are commercially available or can be prepared from aldehydes by methods well known to one skilled in the art. The resulting a-chloroaldoxime can optionally be isolated before it is combined with a compound of Formula IX, wherein R₁ is —CH₂—C≡CH, in the presence of a base such as triethylamine to generate a nitrile oxide in situ and effect the cycloaddition reaction. The reaction with an α-chloroaldoxime can be carried out at room temperature in a suitable solvent such as dichloromethane. Other amines of Formula NH₂—X—CH═CH₂ or NH₂—X—C═C—H can also be used in step (1) of Reaction Scheme Ito provide compounds of Formula I wherein R₁ is a (3-substituted-isoxazol-5-yl)alkyl group or a (3-substituted-4,5-dihydroisoxazol-5-yl)alkyl group.

Synthetic elaboration can also be carried out at the R_{A} or R_{B} position of a compound of Formula I or an intermediate of Formula V through IX. For example, the compounds of Formula V in which R_{B} is a methyl group are known and can be treated according to steps (1) through (4) of Reaction Scheme I to prepare protected 1H-imidazo[4,5-c]pyridin-2-ols of Formula IX. The methyl group at the R_{B} position can then be brominated using N-bromosuccinimide according to the method of Rama Rao, A. V. et al. Tetrahedron Lett., 34, p. 2665, (1993) or Clive, D. L. J. et al. J. Am. Chem. Soc., 116, p. 11275, (1994). The bromine can then be displaced with a variety of primary amines or alkoxide groups using conventional methods, and subsequently step (5) can be followed to provide compounds of Formula I in which R_{B} is a methyl group that is substituted by alkylamino, alkoxy, aryloxy, arylalkyleneoxy, heteroaryloxy, or heteroarylalkyleneoxy.

In another example, the compounds of Formula IX wherein R_{B} is chloro or bromo, which can be prepared using the methods described in Rousseau, R. J., Robins, R. K., J. Heterocycl. Chem., 2, 196 (1965), may be converted to the corresponding compounds wherein R_{B} is alkylamino through palladium-catalyzed coupling with various amines (Wagaw, S., Buchwald, S. L., J. Org. Chem., 61, 7240, (1996)). Likewise, the corresponding compounds wherein R_{B} is alkoxy may be prepared by palladium-catalyzed coupling with the desired alcohol (Palucki, M., Wolfe, J. P., Buchwald, S. L., J. Am. Chem. Soc., 119, 3395, (1997)). Displacement of the 6-chloro group with an alkoxide anion could also provide the corresponding 6-alkoxy derivatives (Japanese Patent No. 04018073 (Tenma et al.)).

The compounds of Formula IX wherein R_{B} is chloro and R_{A} is hydrogen may alternatively by prepared according to Reaction Scheme I, using the compounds of Formula VII wherein R_{B} is chloro and R_{A} is hydrogen. Such compounds of Formula VII can be accessed by reacting a substituted 2,6-dichloro-3-nitropyridin-4-amine of the formula

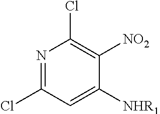

with an amine of the formula (Bn)₂NH. The substituted 2,6-dichloro-3-nitropyridin-4-amine can be prepared by reacting 2,6-dichloro-3-nitropyridin-4-amine with a halogen substituted compound of the formula R₁X in the presence of a base, such as triethylamine. The 2,6-dichloro-3-nitropyridin-4-amine can be prepared by nitrating 2,6-dichloropyridin-4-amine in the presence of concentrated sulfuric acid/nitric acid (10/90) at a reduced temperature, for example, at 0° C. to form 2,6-dichloro-4-nitraminopyridine,

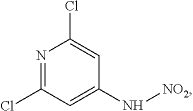

which can be converted to 2,6-dichloro-3-nitropyridin-4-amine in the presence of concentrated sulfuric acid at an elevated temperature, such as heating over a steam bath (Rousseau, R. J., Robins, R. K., J. Heterocycl. Chem., 2, 196 (1965)). The compounds of Formula IX wherein R_{B} is chloro may then be converted to the corresponding compounds wherein R_{B} is alkylamino or alkoxy as described above.

Alternatively, compounds of Formula IX wherein R_{B} is alkoxy can be prepared according to Reaction Scheme I using compounds of Formula VII wherein R_{B} is chloro and R_{A} and R₁ are both hydrogen. The compounds of Formula VII wherein R_{B} is chloro can be converted to the corresponding compounds wherein R_{B} is alkoxy by displacing the chloro group using a metal alkoxide, such as a sodium alkoxide. The reaction can be carried out by adding a 4-amino-6-chloro-3-nitropyridine of Formula VII, where R_{B} is chloro and R_{A} and R₁ are both hydrogen, in a suitable solvent, such as tetrahydrofuran, to a metal alkoxide solution at a reduced temperature, such as at ice bath temperature, and then heating at an elevated temperature, for example, at 85° C. after completing the addition. In step (3) of Reaction Scheme I, the resulting 4-amino-6-alkoxy-3-nitropyridine of Formula VII, where R_{B} is alkoxy and R_{A} and R₁ are both hydrogen, can then be reduced to a 3,4-diamino-6-alkoxypyridine of Formula VIII, where R_{B} is alkoxy and R_{A} and R₁ are both hydrogen. The reduction is conveniently carried out by adding aqueous sodium hydrosulfite to a 4-amino-6-alkoxy-3-nitropyridine of Formula VII in a suitable solvent or solvent mixture such as ethanol/acetonitrile. The reaction can be carried out at room temperature. In step (4) of Reaction Scheme I, a 3,4-diamino-6-alkoxypyridine of Formula VIII can be cyclized to provide a 6-alkoxy-1,3-dihydroimidazo[4,5-c]pyridin-2-one, which is the keto tautomer of Formula IX, where R₁ is hydrogen. The cyclization can be conveniently carried out by heating a 3,4-diamino-6-alkoxypyridine of Formula VIII with 1,1′-carbonyldiimidazole in a suitable solvent such as tetrahydrofuran (THF), tert-butyl methyl ether, dichloromethane, or DMF. The reaction may be carried out at room temperature or, preferably, at an elevated temperature such as the reflux temperature of the solvent. The 1-position of the keto tautomer of Formula IX can be substituted by reaction with a compound of the formula X—R₁ wherein X is a halogen, such as a bromo group, and R₁ is other than hydrogen. The reaction can be carried out by heating a keto tautomer of Formula IX with a compound of formula X—R₁ in a suitable solvent, such as DMF, at an elevated temperature, for example, 80° C. to form a compound of Formula IX, substituted at the 1-position with R₁.

Likewise, compounds of Formula IX wherein R_{B} is alkylamino can be prepared according Scheme I using compounds of Formula VII wherein R_{B} is chloro and R_{A} and R₁ are both hydrogen. The compounds of Formula VII wherein R_{B} is chloro can be converted to the corresponding compounds wherein R_{B} is alkylamino by displacing the chloro group using an excess, such as five equivalents, of an alkylamine, such as, for example, n-butylamine. The reaction can be carried out by adding a 4-amino-6-chloro-3-nitropyridine of Formula VII, where R_{B} is chloro and R_{A} and R₁ are both hydrogen, in a suitable solvent, such as trifluoroethanol, to a solution of the desired alkylamine and then heating at an elevated temperature, for example, at 130° C. in a sealed tube for a period of time, for example, eighteen to twenty-four hours. Steps (3) and (4) of Reaction Scheme I and the installation of an R₁ group other than hydrogen can then be carried out as described above to provide a 1-substituted, 6-alkylamino compound of Formula IX.

In a further alternative, compounds of Formula IX wherein R_{B} is alkoxy may be accessed by O-alkylation of the corresponding 6-oxo-1(3)H-imidazo[4,5-c]pyridin-4-ylamine by O-alkylation methods utilizing a base such as cesium carbonate in a solvent such as DMF (Meurer, L. et al., Bioorg. Med. Chem. Lett., 15(3) 645, (2005)). In addition, O-alkylation could be accomplished under Mitsunobu conditions (Li, Q. et al., Bioorg. Med. Chem. Lett., 16(6), 1679 (2006)).

Step (6) of Reaction Scheme I can be used to prepare a compound of Formula II. The amino group of a pyridine of Formula I can be converted by conventional methods to a functional group such as an amide, carbamate, urea, amidine, or another hydrolyzable group. A compound of this type can be made by the replacement of a hydrogen atom in an amino group with a group such as —C(O)—R′, α-aminoacyl, α-aminoacyl-α-aminoacyl, —C(O)—O—R′, —C(O)—N(R″)—R′, —C(═NY′)—R′, —CH(OH)—C(O)—OY, —CH(OC₁₋₄alkyl)Y₀, —CH₂Y₁, or —CH(CH₃)Y₁; wherein R′ and R″ are each independently C₁₋₁₀ alkyl, C₃₋₇ cycloalkyl, phenyl, or benzyl, each of which may be unsubstituted or substituted by one or more substituents independently selected from the group consisting of halogen, hydroxy, nitro, cyano, carboxy, C₁₋₆ alkyl, C₁₋₄ alkoxy, aryl, heteroaryl, arylC₁₋₄ alkylenyl, heteroarylC₁₋₄ alkylenyl, haloC₁₋₄ alkylenyl, haloC₁₋₄ alkoxy, —O—C(O)—CH₃, —C(O)—O—CH₃, —C(O)—NH₂, —O—CH₂—C(O)—NH₂, —NH₂, and —S(O)₂—NH₂; with the proviso that R″ may also be hydrogen; each α-aminoacyl group is independently selected from racemic, D, or L-amino acids; Y′ is hydrogen, C₁₋₆ alkyl, or benzyl; Y₀ is C₁₋₆ alkyl, carboxyC₁₋₆ alkylenyl, aminoC₁₋₄ alkylenyl, mono-N—C₁₋₆ alkylaminoC₁₋₄ alkylenyl, or di-N,N—C₁₋₆ alkylaminoC₁₋₄ alkylenyl; and Y₁ is mono-N—C₁₋₆ alkylamino, di-N,N—C₁₋₆ alkylamino, morpholin-4-yl, piperidin-1-yl, pyrrolidin-1-yl, or 4-C₁₋₄ alkylpiperazin-1-yl. Particularly useful compounds of Formula II are amides derived from carboxylic acids containing one to ten carbon atoms, amides derived from amino acids, and carbamates containing one to ten carbon atoms. The reaction can be carried out, for example, by combining a compound of Formula I with a chloroformate or acid chloride, such as ethyl chloroformate or acetyl chloride, in the presence of a base such as triethylamine in a suitable solvent such as dichloromethane at room temperature.

Step (6a) of Reaction Scheme I can be used to prepare a compound of Formula III. The hydrogen atom of the alcohol group of Formula I can be replaced using conventional methods with a group such as C₁₋₆ alkanoyloxymethyl, 1-(C₁₋₆ alkanoyloxy)ethyl, 1-methyl-1-(C₁₋₆ alkanoyloxy)ethyl, C₁₋₆ alkoxycarbonyloxymethyl, N—(C₁₋₆ alkoxycarbonyl)aminomethyl, succinoyl, C₁₋₆ alkanoyl, α-aminoC₁₋₄ alkanoyl, arylacyl, —P(O)(OH)₂, —P(O)(O—C₁₋₆ alkyl)₂, C₁₋₆ alkoxycarbonyl, C₁₋₆ alkylcarbamoyl, and α-aminoacyl or α-aminoacyl-α-aminoacyl, where each α-aminoacyl group is independently selected from racemic, D, and L-amino acids. Particularly useful compounds of Formula III are esters made from carboxylic acids containing one to six carbon atoms, unsubstituted or substituted benzoic acid esters, or esters made from naturally occurring amino acids.

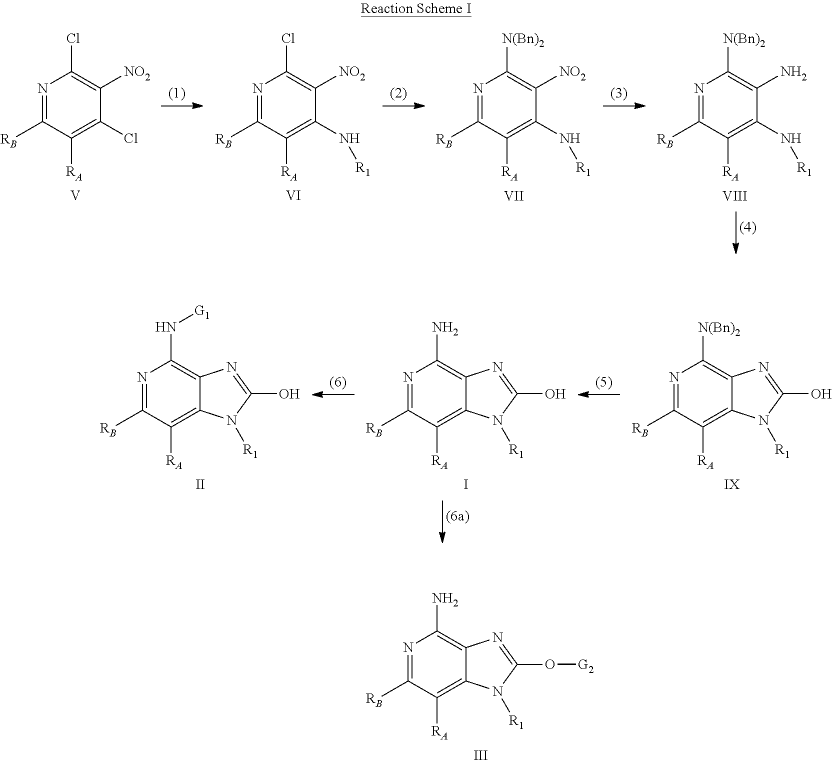

Certain compounds of Formula V can be prepared according to Reaction Scheme 11, wherein R₁₁ and Boc are as defined above, R_{Bx} is alkenyl, —R₁₁, or a carboxy group, and R_{Ba} is alkenyl, —R₁₁ or —NHR₁₁. A 4-hydroxy-2H-pyran-2-one of Formula X in which R_{Bx} is alkenyl or —R₁₁ can be prepared from β,γ-diketoesters according to the method of Lygo, B., Tetrahedron, 51, pp. 12859-12868, (1995) or Song, D. et al., Tetrahedron, 59, pp. 6899-6904, (2003). The compound of Formula X in which R_{B}, is methyl is commercially available and can undergo lithiation-substitution reactions using the method of Poulton, G. A., and Cyr, T. D., Can. J. Chem. 58, p. 2158, (1980) to provide compounds of Formula X in which R_{Bx} is —R₁₁. The compound of Formula X in which R_{Bx} is a carboxy group can be prepared by the method of Stetter, H. and Schellhammer, C.-W., Chem. Ber., 90, p. 755 (1957).

In step (1) of Reaction Scheme II, a 4-hydroxy-2H-pyran-2-one of Formula X is converted to a pyridin-2,4-diol of Formula XI. The reaction can be carried out by heating a compound of Formula X in aqueous ammonium hydroxide at a temperature of 80° C. to 130° C., preferably at a temperature of about 100° C. to about 120° C.

In step (2) of Reaction Scheme II, a compound of Formula XI in which R_{Bx} is a carboxy group is treated with diphenylphosphoryl azide to provide an azide of Formula XII, which undergoes a Curtius rearrangement in step (3) to provide a carbamate-substituted pyridin-2,4-diol of Formula XIII. The Curtius rearrangement in step (3) can be carried out by heating at an elevated temperature such as 70° C. to 110° C. in a suitable solvent such as tert-butanol to provide the tert-butyl carbamate of Formula XIII.

In step (4) of Reaction Scheme II, a carbamate-substituted pyridin-2,4-diol of Formula XIII is deprotected using conventional methods. For example, the Boc group can be removed by treating with trifluoroacetic acid at room temperature to provide an amino-substituted pyridin-2,4-diol of Formula XIV.

In step (5) of Reaction Scheme II, an amino-substituted pyridin-2,4-diol of Formula XIV reacts with an aldehyde or ketone to provide an imine. Numerous aldehydes and ketones are commercially available; others can be readily prepared using known synthetic methods. The reaction can be conveniently carried out by combining the aldehyde or ketone with a compound of Formula XIV in a suitable solvent such as methanol. The reaction can be carried out at room temperature, or at an elevated temperature. Optionally, an acid such as pyridine hydrochloride can be added. The imine is then reduced to provide an amino-substituted of Formula pyridin-2,4-diol of Formula XV. The reduction is conveniently carried out by treating the oxime with excess sodium cyanoborohydride in a suitable solvent or solvent mixture such as methanol/acetic acid. Optionally, hydrochloric acid may be added. The reaction can be carried out at room temperature or at an elevated temperature.

In steps (6) and (7) of Reaction Scheme II, a compound of Formula XI or XV is converted to a 2,4-dichloro-3-nitropyridine of Formula Va by treating first with nitric acid and then with phosphorus(III) oxychloride according to known methods. See, for example, the methods in U.S. Pat. Nos. 5,446,153 (Lindstrom et al.) and 6,525,064 (Dellaria et al.).

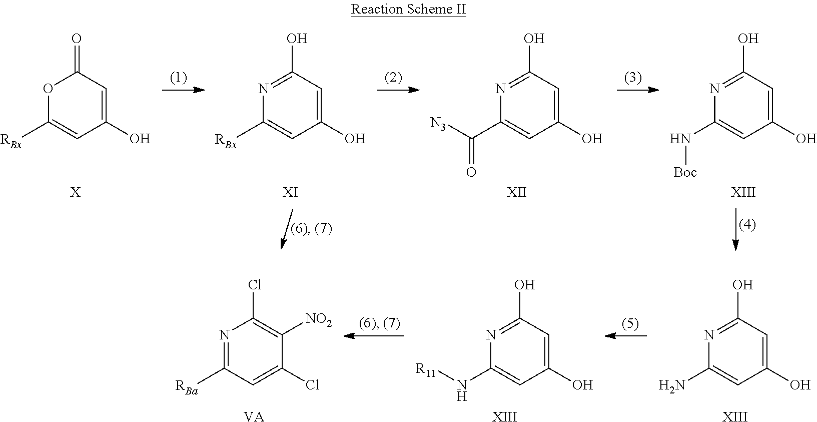

Compounds of the invention can also be prepared using variations of the synthetic routes shown in Reaction Schemes I and II that would be apparent to one of skill in the art, including variations described in the EXAMPLES below.

### Pharmaceutical Compositions and Biological Activity

Pharmaceutical compositions of the invention contain a therapeutically effective amount of a compound or salt of the invention as described above in combination with a pharmaceutically acceptable carrier.

The terms “a therapeutically effective amount” and “effective amount” mean an amount of the compound or salt sufficient to induce a therapeutic or prophylactic effect, such as cytokine induction, cytokine inhibition, immunomodulation, antitumor activity, and/or antiviral activity. The exact amount of compound or salt used in a pharmaceutical composition of the invention will vary according to factors known to those of skill in the art, such as the physical and chemical nature of the compound or salt, the nature of the carrier, and the intended dosing regimen.

In some embodiments, the compositions of the invention will contain sufficient active ingredient or prodrug to provide a dose of about 100 nanograms per kilogram (ng/kg) to about 50 milligrams per kilogram (mg/kg), preferably about 10 micrograms per kilogram (μg/kg) to about 5 mg/kg, of the compound or salt to the subject.

In other embodiments, the compositions of the invention will contain sufficient active ingredient or prodrug to provide a dose of, for example, from about 0.01 mg/m² to about 5.0 mg/m², computed according to the Dubois method, in which the body surface area of a subject (m²) is computed using the subject's body weight: m²=(wt kg^{0.425}×height cm^{0.725})×0.007184, although in some embodiments the methods may be performed by administering a compound or salt or composition in a dose outside this range. In some of these embodiments, the method includes administering sufficient compound to provide a dose of from about 0.1 mg/m² to about 2.0 mg/m² to the subject, for example, a dose of from about 0.4 mg/m² to about 1.2 mg/m².

A variety of dosage forms may be used, such as tablets, lozenges, capsules, parenteral formulations, syrups, creams, ointments, aerosol formulations, transdermal patches, transmucosal patches and the like. These dosage forms can be prepared with conventional pharmaceutically acceptable carriers and additives using conventional methods, which generally include the step of bringing the active ingredient into association with the carrier.

The compounds or salts of the invention can be administered as the single therapeutic agent in the treatment regimen, or the compounds or salts described herein may be administered in combination with one another or with other active agents, including additional immune response modifiers, antivirals, antibiotics, antibodies, proteins, peptides, oligonucleotides, etc.

Compounds or salts of the invention have been shown to induce the production of certain cytokines in experiments performed according to the tests set forth below. These results indicate that the compounds or salts are useful for modulating the immune response in a number of different ways, rendering them useful in the treatment of a variety of disorders.

In some embodiments, compounds or salts of Formula I can be especially useful as immune response modifiers due to their ability to selectively induce IFN-α. As used herein, to “selectively induce IFN-α” means, that when tested according to the test methods described herein, the effective minimum concentration (of the compound or salt) for IFN-α induction is less than the effective minimum concentration for TNF-α induction. In some embodiments, the effective minimum concentration for IFN-α induction is at least 3-fold less than the effective minimum concentration for TNF-α induction. In some embodiments, the effective minimum concentration for IFN-α induction is at least 6-fold less than the effective minimum concentration for TNF-α induction. In other embodiments, the effective minimum concentration for IFN-α induction is at least 10-fold less than the effective minimum concentration for TNF-α induction. In other embodiments, the effective minimum concentration for IFN-α induction is at least 100-fold less than the effective minimum concentration for TNF-α induction. In some embodiments, when tested according to the test methods described herein, the amount TNF-α induced by compounds of the invention is at or below the background level of TNF-α in the test method. Compounds or salts of the invention may, therefore, provide a benefit, for example, a reduced inflammatory response, particularly when administered systemically, over compounds that also induce pro-inflammatory cytokines (e.g. TNF-α) or that induce pro-inflammatory cytokines at higher levels.

Cytokines whose production may be induced by the administration of compounds or salts of the invention generally include interferon-α (IFN-α) and tumor necrosis factor-α (TNF-α) as well as certain interleukins (IL). Cytokines whose biosynthesis may be induced by compounds or salts of the invention include IFN-α, TNF-α, IL-1, IL-6, IL-10 and IL-12, and a variety of other cytokines. Among other effects, these and other cytokines can inhibit virus production and tumor cell growth, making the compounds or salts useful in the treatment of viral diseases and neoplastic diseases. Accordingly, the invention provides a method of inducing cytokine biosynthesis in an animal comprising administering an effective amount of a compound or salt or composition of the invention to the animal. The animal to which the compound or salt or composition is administered for induction of cytokine biosynthesis may have a disease as described infra, for example a viral disease or a neoplastic disease, and administration of the compound or salt may provide therapeutic treatment. Alternatively, the compound or salt may be administered to the animal prior to the animal acquiring the disease so that administration of the compound or salt may provide a prophylactic treatment.

In addition to the ability to induce the production of cytokines, compounds or salts described herein can affect other aspects of the innate immune response. For example, natural killer cell activity may be stimulated, an effect that may be due to cytokine induction. The compounds or salts may also activate macrophages, which in turn stimulate secretion of nitric oxide and the production of additional cytokines. Further, the compounds or salts may cause proliferation and differentiation of B-lymphocytes.

Compounds or salts of the invention can also have an effect on the acquired immune response. For example, the production of the T helper type 1 (T_{H}1) cytokine IFN-γ may be induced indirectly and the production of the T helper type 2 (T_{H}2) cytokines IL-4, IL-5 and IL-13 may be inhibited upon administration of the compounds or salts. Whether for prophylaxis or therapeutic treatment of a disease, and whether for effecting innate or acquired immunity, the compound or salt or composition may be administered alone or in combination with one or more active components as in, for example, a vaccine adjuvant. When administered with other components, the compound or salt and other component or components may be administered separately; together but independently such as in a solution; or together and associated with one another such as (a) covalently linked or (b) non-covalently associated, e.g., in a colloidal suspension.

Conditions for which compounds or salts identified herein may be used as treatments include, but are not limited to:

(a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV);

(b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella;

(c) other infectious diseases, such chlamydia, fungal diseases including but not limited to candidiasis, aspergillosis, histoplasmosis, cryptococcal meningitis, or parasitic diseases including but not limited to malaria, pneumocystis carnii pneumonia, leishmaniasis, cryptosporidiosis, toxoplasmosis, and trypanosome infection;

(d) neoplastic diseases, such as intraepithelial neoplasias, cervical dysplasia, actinic keratosis, basal cell carcinoma, squamous cell carcinoma, renal cell carcinoma, Kaposi's sarcoma, melanoma, leukemias including but not limited to acute myeloid leukemia, acute lymphocytic leukemia, chronic myeloid leukemia, chronic lymphocytic leukemia, multiple myeloma, Hodgkin's lymphoma, non-Hodgkin's lymphoma, cutaneous T-cell lymphoma, B-cell lymphoma, and hairy cell leukemia, and other cancers;

(e) T_{H}2-mediated, atopic diseases, such as atopic dermatitis or eczema, eosinophilia, asthma, allergy, allergic rhinitis, and Ommen's syndrome;

(f) certain autoimmune diseases such as systemic lupus erythematosus, essential thrombocythaemia, multiple sclerosis, discoid lupus, alopecia greata; and

(g) diseases associated with wound repair such as, for example, inhibition of keloid formation and other types of scarring (e.g., enhancing wound healing, including chronic wounds).

Additionally, a compound or salt of the present invention may be useful as a vaccine adjuvant for use in conjunction with any material that raises either humoral and/or cell mediated immune response, such as, for example, live viral, bacterial, or parasitic immunogens; inactivated viral, tumor-derived, protozoal, organism-derived, fungal, or bacterial immunogens; toxoids; toxins; self-antigens; polysaccharides; proteins; glycoproteins; peptides; cellular vaccines; DNA vaccines; autologous vaccines; recombinant proteins; and the like, for use in connection with, for example, BCG, cholera, plague, typhoid, hepatitis A, hepatitis B, hepatitis C, influenza A, influenza B, parainfluenza, polio, rabies, measles, mumps, rubella, yellow fever, tetanus, diphtheria, hemophilus influenza b, tuberculosis, meningococcal and pneumococcal vaccines, adenovirus, HIV, chicken pox, cytomegalovirus, dengue, feline leukemia, fowl plague, HSV-1 and HSV-2, hog cholera, Japanese encephalitis, respiratory syncytial virus, rotavirus, papilloma virus, yellow fever, and Alzheimer's Disease.

Compounds or salts of the present invention may be particularly helpful in individuals having compromised immune function. For example, compounds or salts may be used for treating the opportunistic infections and tumors that occur after suppression of cell mediated immunity in, for example, transplant patients, cancer patients and HIV patients.

Thus, one or more of the above diseases or types of diseases, for example, a viral disease or a neoplastic disease may be treated in an animal in need thereof (having the disease) by administering a therapeutically effective amount of a compound or salt of Formula I, II, III, any of the embodiments described herein, or a combination thereof to the animal.

An animal may also be vaccinated by administering an effective amount of a compound or salt of Formula I, II, III, any of the embodiments described herein, or a combination thereof to the animal as a vaccine adjuvant. In one embodiment, there is provided a method of vaccinating an animal comprising administering an effective amount of a compound or salt described herein to the animal as a vaccine adjuvant.

An amount of a compound or salt effective to induce cytokine biosynthesis is an amount sufficient to cause one or more cell types, such as monocytes, macrophages, dendritic cells and B-cells to produce an amount of one or more cytokines such as, for example, IFN-α, TNF-α, IL-1, IL-6, IL-10 and IL-12 that is increased (induced) over a background level of such cytokines. The precise amount will vary according to factors known in the art but is expected to be a dose of about 100 ng/kg to about 50 mg/kg, preferably about 10 μg/kg to about 5 mg/kg. In other embodiments, the amount is expected to be a dose of, for example, from about 0.01 mg/m² to about 5.0 mg/m², (computed according to the Dubois method as described above) although in some embodiments the induction or inhibition of cytokine biosynthesis may be performed by administering a compound or salt in a dose outside this range. In some of these embodiments, the method includes administering sufficient compound or salt or composition to provide a dose of from about 0.1 mg/m² to about 2.0 mg/m² to the subject, for example, a dose of from about 0.4 mg/m² to about 1.2 mg/m².

The invention also provides a method of treating a viral infection in an animal and a method of treating a neoplastic disease in an animal comprising administering an effective amount of a compound or salt or composition of the invention to the animal. An amount effective to treat or inhibit a viral infection is an amount that will cause a reduction in one or more of the manifestations of viral infection, such as viral lesions, viral load, rate of virus production, and mortality as compared to untreated control animals. The precise amount that is effective for such treatment will vary according to factors known in the art but is expected to be a dose of about 100 ng/kg to about 50 mg/kg, preferably about 10 μg/kg to about 5 mg/kg. An amount of a compound or salt effective to treat a neoplastic condition is an amount that will cause a reduction in tumor size or in the number of tumor foci. Again, the precise amount will vary according to factors known in the art but is expected to be a dose of about 100 ng/kg to about 50 mg/kg, preferably about 10 μg/kg to about 5 mg/kg. In other embodiments, the amount is expected to be a dose of, for example, from about 0.01 mg/m² to about 5.0 mg/m², (computed according to the Dubois method as described above) although in some embodiments either of these methods may be performed by administering a compound or salt in a dose outside this range. In some of these embodiments, the method includes administering sufficient compound or salt to provide a dose of from about 0.1 mg/m² to about 2.0 mg/m² to the subject, for example, a dose of from about 0.4 mg/m² to about 1.2 mg/m².

The methods of the invention may be performed on any suitable subject. Suitable subjects include but are not limited to animals such as but not limited to humans, non-human primates, rodents, dogs, cats, horses, pigs, sheep, goats, or cows.

In addition to the formulations and uses described specifically herein, other formulations, uses, and administration devices suitable for compounds of the present invention are described in, for example, International Publication Nos. WO 03/077944 and WO 02/036592, U.S. Pat. No. 6,245,776, and U.S. Publication Nos. 2003/0139364, 2003/185835, 2004/0258698, 2004/0265351, 2004/076633, and 2005/0009858.

Objects and advantages of this invention are further illustrated by the following examples, but the particular materials and amounts thereof recited in these examples, as well as other conditions and details, should not be construed to unduly limit this invention.

### EXAMPLES

In the examples below automated flash chromatography was carried out using a HORIZON HPFC system (an automated high-performance flash purification product available from Biotage, Inc, Charlottesville, Va., USA). For some of these purifications, either a FLASH 40+M silica cartridge or a FLASH 25+M silica cartridge (both available from Biotage, Inc, Charlottesville, Va., USA) was used. In some chromatographic separations, the solvent mixture 80/18/2 v/v/v chloroform/methanol/concentrated ammonium hydroxide (CMA) was used as the polar component of the eluent. In these separations, CMA was mixed with chloroform in the indicated ratio.

### Preparation of N,N-Bis(4-methoxybenzyl)amine

### Part A

4-Methoxybenzylamine (40 g, 290 mmol) was cooled to 0° C., and p-anisaldehyde (39.7 g, 292 mmol) was added dropwise. The reaction was stirred at ambient temperature for two hours, concentrated under reduced pressure, and further dried under high vacuum overnight to provide 97 g of N-(4-methoxybenzyl)-N-[(4-methoxyphenyl)methylidene]amine as a white, waxy solid.

### Part B

A solution of the material from Part A in ethanol (300 mL) was cooled to 0° C. and stirred rapidly. Solid sodium borohydride (22.1 g, 584 mmol) was added slowly over a period of several minutes, and the reaction was stirred at ambient temperature for two hours. Water (300 mL) was added, and the resulting mixture was shaken and allowed to stand overnight. The mixture was extracted with diethyl ether (3×100 mL), and the combined extracts were washed with water (200 mL), dried over magnesium sulfate, filtered through a layer of CELITE filter agent, concentrated under reduced pressure, and further dried under high vacuum to provide 67 g of N,N-bis(4-methoxybenzyl)amine as a white solid.

### Example 1

### 4-Amino-1-benzyl-6-pentyl-1H-imidazo[4,5-c]pyridin-2-ol

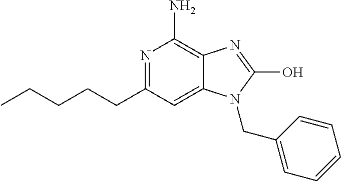

### Part A

A solution of hexanoic acid (2.51 mL, 0.0200 mol) in dichloromethane (5 mL) was cooled to 0° C., and oxalyl chloride (8.7 mL, 0.10 mol) was added. The solution was allowed to warm to room temperature and stirred for 20 hours under a nitrogen atmosphere. The solvent was removed under reduced pressure, and the residue was dissolved in hexane (50 mL). The solution was cooled to 0° C., and triethylamine (3.1 mL, 22 mmol) and 2-methylaziridine (1.57 mL of 90% pure material, 20 mmol) were sequentially added. The resultant mixture was stirred for one hour under a nitrogen atmosphere, diluted with ethyl acetate (50 mL), and filtered through a layer of CELITE filter agent. The filtrate was concentrated under reduced pressure, and the residue was purified by flash chromatography on silica gel (eluting with 11% ethyl acetate in hexane) to provide 1.91 g of 1-hexanoyl-2-methylaziridine as a yellow oil.

### Part B

Sodium hydride (537 mg of a 60% dispersion in mineral oil, 13.4 mmol) was washed three times with hexanes and then suspended in tetrahydrofuran (THF) (30 mL). A solution of tert-butyl acetoacetate (1.94 g, 12.2 mmol) in THF (10 mL) was added dropwise to the suspension, and the mixture was stirred for 30 minutes and then cooled to 0° C. n-Butyllithium (8.4 mL of a 1.6 M solution in hexane) was added, and the resulting yellow-orange solution was stirred at 0° C. for 20 minutes. A solution of 1-hexanoyl-2-methylaziridine (1.90 g, 12.2 mmol) in THF (10 mL) was added, and the reaction was stirred at 0° C. for 1.5 hours. Saturated aqueous ammonium chloride was added, and the mixture was extracted with ethyl acetate (3×40 mL). The combined extracts were dried over magnesium sulfate, filtered, and concentrated under reduced pressure. The crude product was purified by flash chromatography on silica gel (eluting with 5% ethyl acetate in hexane) to provide 1.95 g of tert-butyl 3,5-dioxodecanoate as a colorless oil.

### Part C

Trifluoroacetic acid (16 mL) was added to a solution of tert-butyl 3,5-dioxodecanoate (1.95 g, 7.61 mmol) in dichloromethane (45 mL), and the solution was stirred at room temperature for two hours. The volatiles were removed under reduced pressure, and the residue was dissolved in acetic anhydride (44 mL). The solution was stirred overnight at room temperature, and the acetic anhydride was removed under reduced pressure. The residue was dissolved in methanol (30 mL), and potassium carbonate (105 mg, 0.76 mmol) was added. The mixture was stirred for three hours at room temperature, and an analysis by high-performance liquid chromatography (HPLC) indicated the reaction was incomplete. Additional potassium carbonate (100 mg) was added, and the reaction was stirred for one hour at room temperature. The volatiles were removed under reduced pressure, and the residue was partitioned between saturated aqueous ammonium chloride and dichloromethane. The aqueous layer was separated and extracted with dichloromethane (3×50 mL). The combined organic fractions were dried over magnesium sulfate, filtered, and concentrated under reduced pressure to provide 4-hydroxy-6-pentyl-2H-pyran-2-one as an orange oil that solidified upon standing.

### Part D

A suspension of 4-hydroxy-6-pentyl-2H-pyran-2-one (0.750 g, 4.12 mmol) in concentrated aqueous ammonium hydroxide (10 mL) was heated at 100° C. for six hours and allowed to cool to room temperature. A precipitate was present and was isolated by filtration, triturated with methanol, and isolated by filtration to provide 0.700 g of 4-hydroxy-6-pentylpyridin-2(1B)-one as a tan solid.

### Part E

Fuming nitric acid (20 mL) was carefully added to a suspension of the material from Part D in water (5 mL), and the reaction was heated at 80° C. for 30 minutes, allowed to cool to room temperature, and poured into ice water. Some of the water was removed under reduced pressure, and a precipitate formed. The mixture was cooled to approximately 0° C., and the precipitate was collected by filtration and dried under high vacuum to provide 0.620 g of 3-nitro-6-pentylpyridine-2,4-diol as a pale yellow solid.

### Part F

A solution of 3-nitro-6-pentylpyridine-2,4-diol (1.00 g, 4.42 mmol) in phosphorus(III) oxychloride (15 mL) was heated at 80° C. for four hours. The excess phosphorous(III) oxychloride was removed under reduced pressure, and saturated aqueous sodium bicarbonate was added to adjust the residue to pH 10. The basic mixture was extracted several times with ethyl acetate, and the combined extracts were washed with brine, dried over magnesium sulfate, filtered through a layer of CELITE filter agent, and concentrated under reduced pressure. The crude product was purified by flash chromatography on silica gel (eluting with 25% ethyl acetate in hexane) to provide 0.780 g of 2,4-dichloro-3-nitro-6-pentylpyridine as a tan oil.

### Part G

Triethylamine (1.77 mL, 12.7 mmol) and benzylamine (0.83 mL, 7.6 mmol) were added to a solution of 2,4-dichloro-3-nitro-6-pentylpyridine (2.22 g, 8.44 mmol) in N,N-dimethylformamide (DMF) (50 mL), and the solution was stirred overnight at room temperature. The DMF was removed under reduced pressure, and the residue was partitioned between saturated aqueous sodium bicarbonate and dichloromethane. The aqueous layer was separated and extracted with dichloromethane, and the combined organic fractions were washed with brine, dried over magnesium sulfate, filtered, and concentrated under reduced pressure. The crude product was purified by flash chromatography on silica gel (eluting with 1% to 3% ethyl acetate in hexanes) to provide 1.39 g of N-benzyl-2-chloro-3-nitro-6-pentylpyridin-4-amine.

### Part H

Triethylamine (1.44 mL, 10.3 mmol) and N,N-bis(4-methoxybenzyl)amine (2.65 g, 10.3 mmol) were added to a solution of N-benzyl-2-chloro-3-nitro-6-pentylpyridin-4-amine (2.30 g, 6.89 mmol) in toluene (100 mL), and the yellow solution was heated at reflux overnight under a nitrogen atmosphere. The volatiles were removed under reduced pressure, and the residue was partitioned between ethyl acetate and saturated aqueous sodium bicarbonate. The aqueous layer was separated and extracted with ethyl acetate, and the combined organic fractions were washed with brine, dried over magnesium sulfate, filtered, and concentrated under reduced pressure to provide N⁴-benzyl-N²,N²-bis(4-methoxybenzyl)-3-nitro-6-pentylpyridine-2,4-diamine, which was used without purification.

### Part I

Sodium borohydride (0.150 g, 3.97 mmol) was added to a mixture of nickel(II) chloride hexahydrate (0.820 g, 3.45 mmol) in methanol (60 mL), and the mixture was stirred for 15 minutes. A solution of N⁴-benzyl-N²,N²-bis(4-methoxybenzyl)-3-nitro-6-pentylpyridine-2,4-diamine (3.82 g, 6.89 mmol) in methanol (50 mL) and dichloromethane (25 mL) was added. Additional sodium borohydride (0.319 g, 8.43 mmol) was added in portions over a period of ten minutes, and the mixture was stirred for one hour at room temperature. An analysis by HPLC indicated the presence of starting material, and additional nickel(II) chloride hexahydrate (0.800 g, 3.37 mmol) and sodium borohydride (0.250 g, 6.61 mmol) were added. The reaction was stirred at room temperature for two hours and then filtered through a layer of CELITE filter agent. The filter cake was washed with dichloromethane, and the filtrate was concentrated under reduced pressure. The residue was partitioned between saturated aqueous sodium bicarbonate and dichloromethane, and the work-up procedure described in Part G was followed. The crude product was purified by flash chromatography on silica gel (eluting with 2% to 4% methanol in dichloromethane) to provide 3.45 g of N⁴-benzyl-N²,N²-bis(4-methoxybenzyl)-6-pentylpyridine-2,3,4-triamine as a thick, dark oil.

### Part J

Carbonyl diimidazole (1.60 g, 9.86 mmol) was added to a solution of N⁴-benzyl-N²,N²-bis(4-methoxybenzyl)-6-pentylpyridine-2,3,4-triamine (3.45 g, 6.58 mmol) in THF (50 mL), and the dark green solution was heated at reflux under a nitrogen atmosphere for two hours. The volatiles were removed under reduced pressure, and the residue was purified by flash chromatography on silica gel (eluting with 2% methanol in dichloromethane) to provide 3.52 g of 1-benzyl-4-[bis(4-methoxybenzyl)amino]-6-pentyl-1H-imidazo[4,5-c]pyridin-2-ol as a thick, yellow oil that solidified upon standing.

### Part K

A solution of 1-benzyl-4-[bis(4-methoxybenzyl)amino]-6-pentyl-1H-imidazo[4,5-c]pyridin-2-ol (3.52 g, 6.39 mmol) in trifluoroacetic acid (15 mL) was stirred at room temperature for five hours and then diluted with water. The resulting mixture was adjusted to approximately pH 9 with the addition of solid sodium carbonate. The aqueous layer was separated and extracted several times with dichloromethane and dichloromethane/methanol. The combined organic fractions were dried over magnesium sulfate, filtered, and concentrated under reduced pressure. The resulting white solid was triturated with acetonitrile/methanol and isolated by filtration to provide 1.08 g of 4-amino-1-benzyl-6-pentyl-1H-imidazo[4,5-c]pyridin-2-ol as a white crystalline solid, mp 260-262° C.

¹H NMR (300 MHz, d₄-MeOH) δ 7.57-7.51 (m, 5H), 6.57 (s, 1H), 5.26 (s, 2H), 2.77 (dd, J=7.4, 7.8 Hz, 2H), 1.82 (m, 2H), 1.53-1.48 (m, 4H), 1.10 (t, J=7.0 Hz, 3H);

MS (APCI) m/z 311 (M+H⁺);

Anal. Calcd for C₁₈H₂₂N₄O.0.67 CF₃CO₂H: C, 60.06; H, 5.91; N, 14.48. Found: C, 59.72; H, 6.30; N, 14.57.

### Example 2

### 4-Amino-6-pentyl-1-(2-phenylethyl)-1H-imidazo[4,5-c]pyridin-2-ol

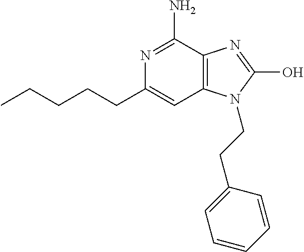

### Part A

Phenethylamine (0.86 mL, 6.8 mmol) was added to a stirred solution of 2,4-dichloro-3-nitro-6-pentylpyridine (2.0 g, 7.6 mmol) and triethylamine (1.6 mL, 11 mmol) in DMF (38 mL), and the reaction was stirred for three hours at room temperature. Water (200 mL) was added, and the mixture was extracted with ethyl acetate (2×50 mL). The combined organic fractions were washed with brine (50 mL), dried over sodium sulfate, filtered, and concentrated under reduced pressure. The crude product was purified by column chromatography on silica gel (eluting with 10% ethyl acetate in hexane) to provide 1.1 g of (2-chloro-3-nitro-6-pentylpyridin-4-yl)phenethylamine as a yellow oil.

### Part B

Triethylamine (0.66 mL, 4.7 mmol) and N,N-bis(4-methoxybenzyl)amine (1.2 g, 4.7 mmol) were added to a solution of (2-chloro-3-nitro-6-pentylpyridin-4-yl)phenethylamine (1.1 g, 3.2 mmol) in toluene (32 mL), and the yellow solution was heated at reflux for three hours, stirred overnight at room temperature, and heated at reflux for two hours. The work-up procedure described in Part H of Example 1 was followed to provide N²,N²-bis(4-methoxybenzyl)-3-nitro-6-pentyl-N⁴-(2-phenylethyl)pyridine-2,4-diamine as an oil.

### Part C

Sodium borohydride (0.070 g, 1.85 mmol) was added to a mixture of nickel(II) chloride hexahydrate (0.38 g, 1.6 mmol) in methanol (25 mL), and the mixture was stirred for 15 minutes. A solution of the material from Part B in methanol (25 mL) and dichloromethane (11 mL) was added. Additional sodium borohydride (0.150 g, 3.97 mmol) was added in portions, and the mixture was stirred for one hour at room temperature. An analysis by thin layer chromatography (TLC) indicated the presence of starting material, and additional sodium borohydride (0.10 g, 2.6 mmol) was added. The reaction was stirred at room temperature overnight. The reaction was still incomplete, and sodium borohydride was added in portions (0.10 g and 0.20 g) until the starting material was consumed. The reaction mixture was filtered through a layer of CELITE filter agent. The filter cake was washed with dichloromethane until the filtrate was colorless, and the filtrate was then concentrated under reduced pressure. The crude product was purified by flash chromatography on silica gel (eluting sequentially with 30% ethyl acetate in hexane and 50% ethyl acetate in hexane) to provide 0.40 g of N²,N²-bis(4-methoxybenzyl)-6-pentyl-N⁴-(2-phenylethyl)pyridine-2,3,4-triamine as a yellow oil.

### Part D

Carbonyl diimidazole (0.18 g, 1.1 mmol) was added to a solution of N²,N²-bis(4-methoxybenzyl)-6-pentyl-N⁴-(2-phenylethyl)pyridine-2,3,4-triamine (0.40 g, 0.74 mmol) in THF (4 mL), and the orange solution was heated at reflux for one hour and allowed to cool to room temperature. The volatiles were removed under reduced pressure, and the residue was purified by flash chromatography on silica gel (eluting sequentially with 30% ethyl acetate in hexane and 50% ethyl acetate in hexane) to provide 0.36 g of 4-[bis(4-methoxybenzyl)amino]-6-pentyl-1-(2-phenylethyl)-1H-imidazo[4,5-c]pyridin-2-ol as an oil that solidified upon standing.

### Part E

A solution of 4-[bis(4-methoxybenzyl)amino]-6-pentyl-1-(2-phenylethyl)-1H-imidazo[4,5-c]pyridin-2-ol (0.36 g, 0.64 mmol) in trifluoroacetic acid (1.6 mL) was stirred at room temperature for five hours and then diluted with water (20 mL). The resulting mixture was adjusted to approximately pH 13 with the addition of aqueous sodium hydroxide (50% w/w) and stirred for one hour. A solid was present and was collected by filtration, washed with water, and recrystallized from acetonitrile (20 mL) and ethanol (5 mL). The crystals were washed with acetonitrile and dried under vacuum for 18 hours at 65° C. to provide 0.10 g of 4-amino-6-pentyl-1-(2-phenylethyl)-1H-imidazo[4,5-c]pyridin-2-ol as a white crystalline powder, mp 238-240° C.

¹H NMR (300 MHz, DMSO-d₆) δ 10.10 (bs, 1H), 7.28-7.17 (m, 5H), 6.24 (s, 1H), 5.50 (bs, 2H), 3.92 (t, J=7.5 Hz, 2H) 2.90 (t, J=7.5 Hz, 2H), 2.44 (t, J=7.5 Hz, 2H), 1.55 (pentet, J=7.5 Hz, 2H), 1.31-1.21 (m, 4H), 0.87 (t, J=7.5 Hz, 3H);

MS (APCI) m/z 325 (M+H)⁺;

Anal. Calcd for C₁₉H₂₄N₄O: C, 70.34; H, 7.46; N, 17.27. Found: C, 70.21; H, 7.50; N, 17.31.

### Example 3

### 4-Amino-1-(2-hydroxy-2-methylpropyl)-6-pentyl-1H-imidazo[4,5-c]pyridin-2-ol

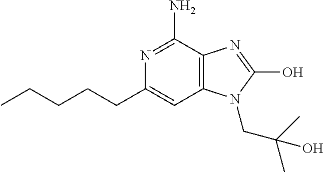

### Part A

1-Amino-2-methylpropan-2-ol (0.64 g, 7.2 mmol) was added to a stirred solution of 2,4-dichloro-3-nitro-6-pentylpyridine (2.1 g, 8.0 mmol) and triethylamine (1.7 mL, 12 mmol) in DMF (40 mL). The reaction was stirred for 18 hours at room temperature and partitioned between water (200 mL) and ethyl acetate (50 mL). The organic layer was separated and washed with brine (50 mL), dried over sodium sulfate, filtered, and concentrated under reduced pressure. The crude product was purified by column chromatography on silica gel (eluting with 30% ethyl acetate in hexane) to provide 1.1 g of 14(2-chloro-3-nitro-6-pentylpyridin-4-yl)amino]-2-methylpropan-2-ol as a bright yellow oil.

### Part B

1-[(2-Chloro-3-nitro-6-pentylpyridin-4-yl)amino]-2-methylpropan-2-ol (1.1 g, 3.5 mmol), triethylamine (0.7 mL, 5 mmol), and N,N-bis(4-methoxybenzyl)amine (1.3 g, 5.2 mmol) were reacted according to the method of Part B of Example 2 with the modification that the reaction was heated at reflux for six hours in toluene (35 mL) and then stirred at room temperature overnight to provide 1-({2-[bis(4-methoxybenzyl)amino]-3-nitro-6-pentylpyridin-4-yl}amino)-2-methylpropan-2-ol after the work-up procedure.

### Part C

Sodium borohydride (0.100 g, 2.64 mmol) was added to a mixture of nickel(II) chloride hexahydrate (0.41 g, 1.7 mmol) in methanol (30 mL), and the mixture was stirred for 15 minutes. A solution of the material from Part B in methanol (28 mL) and dichloromethane (12 mL) was added. Additional sodium borohydride (0.140 g, 3.70 mmol) was added in portions, and the mixture was stirred for one hour at room temperature. An analysis by, TLC indicated the presence of starting material, and additional sodium borohydride was added in portions (0.12 g and 0.12 g). The reaction mixture was stirred briefly and then filtered through a layer of CELITE filter agent. The filter cake was washed with dichloromethane until the filtrate was colorless, and the filtrate was then concentrated under reduced pressure. The crude product was stirred with dichloromethane and filtered again through CELITE filter agent. The filtrate was concentrated under reduced pressure to provide 1-({3-amino-2-[bis(4-methoxybenzyl)amino]-6-pentylpyridin-4-yl}amino)-2-methylpropan-2-ol as a green oil.

### Part D

The material from Part C was treated with carbonyl diimidazole (0.85 g, 5.2 mmol) according to the method described in Part D of Example 2 with the modification that chromatographic purification was carried out eluting sequentially with 50% ethyl acetate in hexane and then ethyl acetate. 4-[Bis(4-methoxybenzyl)amino]-1-(2-hydroxy-2-methylpropyl)-6-pentyl-1H-imidazo[4,5-c]pyridin-2-ol (1.4 g) was obtained as a colorless oil.

### Part E

A solution of 4-[bis(4-methoxybenzyl)amino]-1-(2-hydroxy-2-methylpropyl)-6-pentyl-1H-imidazo[4,5-c]pyridin-2-ol (1.4 g, 2.6 mmol) in trifluoroacetic acid (7 mL) was stirred at room temperature for three hours and then diluted with water (20 mL). The resulting mixture was adjusted to approximately pH 13 with the addition of aqueous sodium hydroxide (50% w/w) and stirred for one hour. A solid was present and was collected by filtration and washed with water to provide 0.8 g of a white solid. The filtrate was allowed to stand for three days, and additional solid formed. The second solid was isolated by filtration and washed with water. The first solid was purified by column chromatography on silica gel (eluting with 10% methanol in dichloromethane) and then combined with the second solid. The combined solids were recrystallized from acetonitrile. The crystals were washed with acetonitrile and dried under vacuum for 17 hours at 65° C. to provide 0.35 g of 4-amino-1-(2-hydroxy-2-methylpropyl)-6-pentyl-1H-imidazo[4,5-c]pyridin-2-ol as white, crystalline plates, mp 240-243° C.

¹H NMR (300 MHz, DMSO-d₆) δ 10.16 (bs, 1H), 6.48 (s, 1H), 5.53 (bs, 2H), 4.58 (s, 3.59 (bs, 2H), 2.47 (t, J=7.5 Hz, 2H), 1.59 (pentet, J=7.5 Hz, 2H), 1.31-1.24 (m, 4H), 1.11 (bs, 6H), 0.85 (t, J=7.5 Hz, 3H);

MS (APCI) m/z 293 (M+H)⁺;

Anal. Calcd for C₁₅H₂₄N₄O₂: C, 61.62; H, 8.27; N, 19.16. Found: C, 61.49; H, 8.57; N, 19.25.

### Example 4

### 4-Amino-6-pentyl-1-(tetrahydro-2H-pyran-4-ylmethyl)-1H-imidazo[4,5-c]pyridin-2-ol

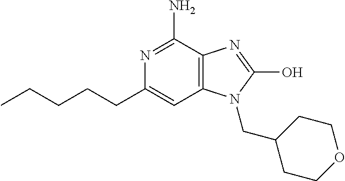

### Part A

Solid tetrahydro-2H-pyran-4-ylmethylamine hydrochloride (see U.S. Patent Application Publication No. 2004/0147543 (Hays et al.) Examples 477-480, 0.78 g, 5.1 mmol) was added to a stirred solution of 2,4-dichloro-3-nitro-6-pentylpyridine (1.5 g, 5.7 mmol) and triethylamine (2.0 mL, 14 mmol) in DMF (29 mL). The reaction was stirred for 18 hours at room temperature. The work-up and purification procedures described in Part A of Example 3 were followed to provide 1.0 g of 2-chloro-3-nitro-6-pentyl-N-(tetrahydro-2H-pyran-4-ylmethyl)pyridin-4-amine.

### Part B

2-Chloro-3-nitro-6-pentyl-N-(tetrahydro-2H-pyran-4-ylmethyl)pyridin-4-amine (1.0 g, 2.9 mmol), triethylamine (0.61 mL, 4.4 mmol), and N,N-bis(4-methoxybenzyl)amine (1.1 g, 4.4 mmol) were reacted according to the method of Part B of Example 2 with the modification that the reaction was heated at reflux for 14 hours in toluene (29 mL) to provide N²,N²-bis(4-methoxybenzyl)-3-nitro-6-pentyl-N⁴-(tetrahydro-2H-pyran-4-ylmethyl)pyridine-2,4-diamine as a yellow oil after the work-up procedure.

### Part C

The method described in Part C of Example 3 was used to reduce the material from Part B to N²,N²-bis(4-methoxybenzyl)-6-pentyl-N⁴-(tetrahydro-2H-pyran-4-ylmethyl)pyridine-2,3,4-triamine, which was obtained as a dark green oil.

### Part D

The material from Part C was treated with carbonyl diimidazole (0.71 g, 4.4 mmol) according to the method described in Part D of Example 2 with the modification that chromatographic purification was carried out using an automated flash chromatography system with a 40+M silica cartridge and eluting with 40% to 80% ethyl acetate in hexane. 4-[Bis(4-methoxybenzyl)amino]-6-pentyl-1-(tetrahydro-2H-pyran-4-ylmethyl)-1H-imidazo[4,5-c]pyridin-2-ol (0.75 g) was obtained as an oil that solidified upon standing.

### Part E

A solution of 4-[bis(4-methoxybenzyl)amino]-6-pentyl-1-(tetrahydro-2H-pyran-4-ylmethyl)-1H-imidazo[4,5-c]pyridin-2-ol (0.75 g, 1.3 mmol) in trifluoroacetic acid (3 mL) was stirred at room temperature for 18 hours and then diluted with water (20 mL). The resulting mixture was adjusted to approximately pH 13 with the addition of aqueous sodium hydroxide (50% w/w) and stirred for one hour. A solid was present and was collected by filtration and washed with water to provide 0.5 g of a white solid. The filtrate was allowed to stand for three days, and additional solid formed. The second solid was isolated by filtration. The first solid was purified by automated flash chromatography (25+M silica cartridge, eluting with 0% to 15% methanol in dichloromethane) and then combined with the second solid. The combined solids (0.27 g) were recrystallized from acetonitrile (50 mL) and ethanol (8 mL). The crystals were washed with acetonitrile and dried under vacuum for 17 hours at 65° C. to provide 0.24 g of 4-amino-6-pentyl-1-(tetrahydro-2H-pyran-4-ylmethyl)-1H-imidazo[4,5-c]pyridin-2-ol as white, crystalline plates, mp>250° C.

¹H NMR (300 MHz, DMSO-d₆) δ 10.13 (bs, 1H), 6.43 (s, 1H), 5.54 (bs, 2H), 3.83-3.79 (m, 2H), 3.58 (d, J=7.5 Hz, 2H), 3.25-3.18 (m, 2H), 2.50-2.46 (m, 2H), 1.99-1.94 (m, 1H), 1.60 (pentet, J=7.5 Hz, 2H), 1.47-1.43 (m, 2H), 1.32-1.22 (m, 6H), 0.86 (t, J=6.9 Hz, 3H);

MS (APCI) m/z 319 (M+H)⁺;

Anal. Calcd for C₁₇H₂₆N₄O₂; C, 64.13; H, 8.23; N, 17.60. Found: C, 64.08; H, 8.11; N, 17.64.

### Example 5

### 4-Amino-6-pentyl-1-(pyridin-3-ylmethyl)-1H-imidazo[4,5-c]pyridin-2-ol

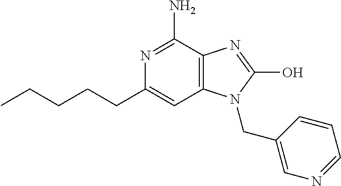

### Part A

3-(Aminomethyl)pyridine (0.52 mL, 5.1 mmol) was added to a stirred solution of 2,4-dichloro-3-nitro-6-pentylpyridine (1.5 g, 5.7 mmol) and triethylamine (2.0 mL, 14 mmol) in DMF (29 mL), and the methods described in Part A of Example 3 were followed with the modification that the crude product was purified by automated flash chromatography (40+M cartridge, eluting with 0% to 30% ethyl acetate in hexane) to provide 1.0 g of 2-chloro-3-nitro-6-pentyl-N-(pyridin-3-ylmethyl)pyridin-4-amine as a yellow oil.

### Part B

Triethylamine (0.63 mL, 4.5 mmol) and N,N-bis(4-methoxybenzyl)amine (1.2 g, 4.5 mmol) were added to a solution of 2-chloro-3-nitro-6-pentyl-N-(pyridin-3-ylmethyl)pyridin-4-amine (1.0 g, 3.0 mmol) in toluene (30 mL), and the yellow solution was heated at reflux for 14 hours, allowed to cool to room temperature, and concentrated under reduced pressure to provide N²,N²-bis(4-methoxybenzyl)-3-nitro-6-pentyl-N⁴-(pyridin-3-ylmethyl)pyridine-2,4-diamine.

### Part C

Sodium borohydride (0.20 g, 5.4 mmol) was added to a mixture of nickel(II) chloride hexahydrate (0.36 g, 1.5 mmol) in methanol (20 mL), and the mixture was stirred for 15 minutes. A solution of the material from Part B in methanol (50 mL) and dichloromethane (11 mL) was added, and the reaction was stirred for 30 minutes. Additional sodium borohydride (0.20 g, 5.4 mmol) was added, and an analysis by TLC indicated the presence of starting material. Additional sodium borohydride was added (0.10 g), and then the reaction was complete. The reaction mixture was stirred briefly and then filtered through a layer of CELITE filter agent. The filter cake was washed with dichloromethane until the filtrate was colorless, and the filtrate was then concentrated under reduced pressure. The crude product was stirred with dichloromethane and filtered again through CELITE filter agent. The filtrate was concentrated under reduced pressure to provide N²,N²-bis(4-methoxybenzyl)-6-pentyl-N⁴-(pyridin-3-ylmethyl)pyridine-2,3,4-triamine as a green oil.

### Part D

The material from Part C was treated with carbonyl diimidazole (0.73 g, 4.5 mmol) according to the method described in Part D of Example 2 with the modification that chromatographic purification was carried out using an automated flash chromatography system with a 40+M silica cartridge and eluting with a gradient of 70% ethyl acetate in hexane to 100% ethyl acetate. 4-[Bis(4-methoxybenzyl)amino]-6-pentyl-1-(pyridin-3-ylmethyl)-1H-imidazo[4,5-c]pyridin-2-ol (1.3 g) was obtained as an oil that solidified somewhat upon standing.

### Part E

A solution of 4-[bis(4-methoxybenzyl)amino]-6-pentyl-1-(pyridin-3-ylmethyl)-1H-imidazo[4,5-c]pyridin-2-ol (1.3 g, 2.4 mmol) in trifluoroacetic acid (6 mL) was stirred at room temperature for two hours and then diluted with water (20 mL). The resulting mixture was adjusted to approximately pH 13 with the addition of aqueous sodium hydroxide (50% w/w) and stirred for two hours. A solid was present and was collected by filtration and washed with water to provide 1 g of a white solid. The solid was purified by automated flash chromatography (40+M silica cartridge, eluting with 0% to 20% methanol in dichloromethane) followed by recrystallization from ethanol (10 mL). The crystals were washed with ethanol and dried under vacuum for four hours at 65° C. to provide 4-amino-6-pentyl-1-(pyridin-3-ylmethyl)-1H-imidazo[4,5-c]pyridin-2-ol as a white crystalline powder, mp 243-245° C.

¹H NMR (300 MHz, DMSO-d₆) δ 10.28 (bs, 1H), 8.57 (d, J=2.5 Hz, 1H), 8.48-8.47 (m, 1H), 7.68-7.66 (m, 1H), 7.37-7.34 (m, 1H), 6.41 (s, 1H), 5.59 (bs, 2H), 4.96 (bs, 2H), 2.45 (t, J=7.5 Hz, 2H), 1.56 (pentet, J=7.5 Hz, 2H), 1.28-1.19 (m, 4H), 0.83 (t, J=6.9 Hz, 3H);

MS (APCI) m/z 312 (M+H)⁺;

Anal. Calcd for C₁₇H₂₁N₅O.0.25 H₂O: C, 64.64; H, 6.86; N, 22.17. Found: C, 64.32; H, 7.14; N, 22.16.

### Example 6

### 4-Amino-1-benzyl-6-methyl-1H-imidazo[4,5-c]pyridin-2-ol

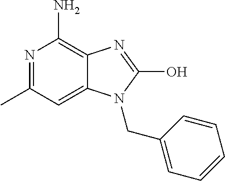

4-Amino-1-benzyl-6-methyl-1H-imidazo[4,5-c]pyridin-2-ol was prepared according to the general methods described in Parts G through K of Example 1 using 2,4-dichloro-6-methyl-3-nitropyridine in lieu of 2,4-dichloro-3-nitro-6-pentylpyridine in Part G. The crude product was purified by automated flash chromatography (40+M cartridge, eluting with 0% to 15% methanol in dichloromethane) followed by recrystallization from methanol to provide 4-amino-1-benzyl-6-methyl-1H-imidazo[4,5-c]pyridin-2-ol as a crystalline solid, mp>300° C. Anal. Calcd for C₁₄H₁₄N₄O: C, 66.13; H, 5.55; N, 22.03. Found: C, 66.22; H, 5.33; N, 22.03.

### Example 7

### 4-Amino-6-pentyl-1-{2-[3-[(1,3-thiazolo-2-yl)propoxy]ethyl}-1H-imidazo[4,5-c]pyridin-2-ol

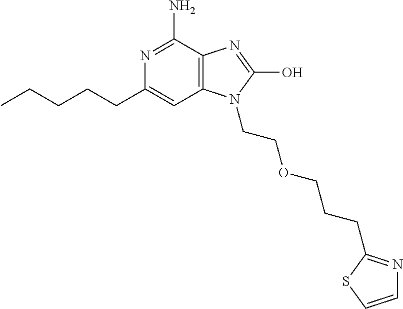

4-Amino-6-pentyl-1-{2-[3-[(1,3-thiazolo-2-yl)propoxy]ethyl}-1H-imidazo[4,5-c]pyridin-2-ol was prepared according to the general methods described in Parts A through E of Example 2 using 2-[3-[(1,3-thiazolo-2-yl)propoxy]ethylamine (see U.S. Pat. No. 6,797,718 (Dellaria et al.) Example 82) in lieu of phenethylamine in Part A. The crude product was purified by automated flash chromatography (40+M cartridge, eluting with 0% to 20% methanol in dichloromethane) followed by recrystallization from acetonitrile to provide 4-amino-6-pentyl-1-{2-[3-[(1,3-thiazolo-2-yl)propoxy]ethyl}-1H-imidazo[4,5-e]pyridin-2-ol as a crystalline solid, mp 177.0-178.0° C. Anal. Calcd for C₁₉H₂₇N₅O₂S: C, 58.59; H, 6.99; N, 17.98. Found: C, 58.66; H, 6.86; N, 18.03.

### Example 8

### N-[3-(4-Amino-2-hydroxy-6-pentyl-1H-imidazo[4,5-c]pyridin-1-yl)propyl]methanesulfonamide

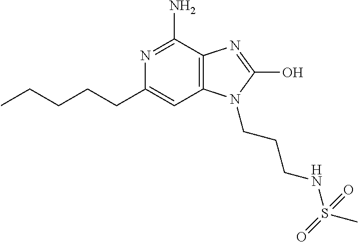

### Part A

Tert-butyl 3-aminopropylcarbamate (2.4 g, 13.68 mmol) was added to a solution of 2,4-dichloro-3-nitro-6-pentylpyridine (4 g, 15.2 mmol) and triethylamine (3.8 g, 38.0 mmol) in DMF (76 mL). The reaction mixture was stirred at ambient temperature for 18 hours and then partitioned between ethyl acetate (50 mL) and water (200 mL). The organic layer was washed with brine (50 mL), dried over sodium sulfate, filtered, and then concentrated under reduced pressure to provide crude product as a bright yellow oil. This material was purified by automated flash chromatography (40+M cartridge, eluting with 10% to 50% ethyl acetate in hexanes) to provide 3 g of tert-butyl 3-[(2-chloro-3-nitro-6-pentylpyridin-4-yl)amino]propylcarbamate as a yellow oil.

### Part B

A solution of the material from Part A (3 g, 7.48 mmol), N,N-bis(4-methoxybenzyl)amine (2.9 g, 11.22 mmol), and triethylamine (1.1 g, 11.22 mmol) in toluene (75 mL) was heated at reflux for 18 hours and then concentrated under reduced pressure to provide 4.6 g of crude tert-butyl 3-({2-[bis(4-methoxybenzyl)amino]-3-nitro-6-pentylpyridin-4-yl}amino)propylcarbamate.

### Part C

Solid sodium borohydride (0.25 g, 6.6 mmol) was added in a single portion to a solution of nickel(II) chloride hexahydrate (0.9 g, 3.7 mmol) in methanol (50 mL) and the resulting suspension was stirred for 15 minutes. A solution of the material from Part B (about 7.5 mmol) in a mixture of dichloromethane (27 mL) and methanol (75 mL) was added in a single portion to the suspension. Sodium borohydride (0.26 g, 6.9 mmol) was added. After 30 minutes more sodium borohydride (0.2 g, 5 mmol) was added. Small portions of sodium borohydride were added until analysis by thin layer chromatography indicated that all of the starting material had been consumed. The reaction mixture was filtered through a layer of CELITE filter agent and the filter cake was washed with dichloromethane until the wash was clear. The filtrate was concentrated under reduced pressure to provide about 4.4 g of tert-butyl 3-({3-amino-2-[bis(4-methoxybenzyl)amino]-6-pentylpyridin-4-yl}amino)propylcarbamate as a green oil.

### Part D

A solution of the material from Part C (about 7.5 mmol) and carbonyldiimdazole (1.8 g, 11 mmol) in THF (37 mL) was heated at reflux for 1 hour, cooled to ambient temperature, and then concentrated under reduced pressure to provide crude product. This material was purified by automated flash chromatography (40+M cartridge, eluting with 70% to 100% ethyl acetate in hexanes) to provide 3 g of tert-butyl 3-{4-[bis(4-methoxybenzyl)amino]-2-hydroxy-6-pentyl-1H-imidazo[4,5-c]pyridin-1-yl}propylcarbamate as a solid.

### Part E

A solution of the material from Part D in trifluoroacetic acid (12 mL) was stirred at ambient temperature for 2 hours. Water (20 mL) was added and a white precipitate formed. The pH was adjusted to about 13 with 50% sodium hydroxide and the suspension was stirred at ambient temperature for 96 hours. The solid was removed by filtration and the filter cake was rinsed with water. The filtrate was concentrated under reduced pressure to provide a solid. This material was slurried with chloroform (100 mL) for 1 hour. The solid was removed by filtration. The filtrate was concentrated under reduced pressure to provide crude product as a brown foam. The foam was purified by automated flash chromatography (40+M cartridge, eluting with 30% to 60% CMA in chloroform) to provide 0.6 g of 4-amino-1-(3-aminopropyl)-6-pentyl-1H-imidazo[4,5-c]pyridin-2-ol as a white, waxy solid.

### Part F

Methanesulfonyl chloride (0.12 g, 1.08 mmol) was added to a suspension of material from Part E (0.25 g, 0.90 mmol) in triethylamine (0.31 mL) and dichloromethane (5 mL). The resulting solution was stirred at ambient temperature for 1 hour and then partitioned between dichloromethane (50 mL) and saturated aqueous ammonium chloride (50 mL). The organic layer was dried over sodium sulfate, filtered, and then concentrated under reduced pressure to provide crude product as a solid. This material was purified by automated flash chromatography (25+M cartridge, eluting with 0% to 10% methanol in dichloromethane) followed by recrystallization from a mixture of acetonitrile (15 mL) and ethanol (2 mL) to provide 0.2 g of N-[3-(4-amino-2-hydroxy-6-pentyl-1H-imidazo[4,5-c]pyridin-1-yl)propyl]methanesulfonamide as crystalline solid, mp 174.0-176.0° C. Anal. Calcd for C₁₅H₂₅N₅O₃S: C, 50.68; H, 7.09; N, 19.70. Found: C, 50.76; H, 7.42; N, 19.85.

### Example 9

### N-[3-(4-Amino-2-hydroxy-6-pentyl-1-imidazo[4,5-c]pyridin-1-yl)propyl]-N′-cyclohexylurea

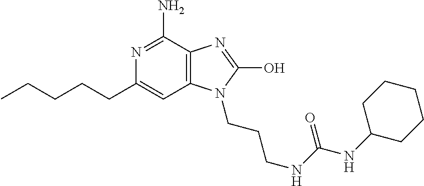

Cyclohexyl isocyanate (0.1 g, 0.8 mmol) was added to a suspension of 4-amino-1-(3-aminopropyl)-6-pentyl-1H-imidazo[4,5-c]pyridin-2-ol (0.2 g, 0.7 mmol) in dichloromethane (4 mL). The resulting solution was stirred at ambient temperature for 2 hours and then concentrated under reduced pressure to provide crude product. This material was purified by automated flash chromatography (25+M cartridge, eluting with 0% to 10% methanol in dichloromethane) followed by recrystallization from a mixture of acetonitrile (15 mL) and ethanol (8 mL) to provide 0.2 g of N-[3-(4-amino-2-hydroxy-6-pentyl-1H-imidazo[4,5-c]pyridin-1-yl)propyl]-N′-cyclohexylurea as a crystalline solid, mp 202.0-204.0° C. Anal. Calcd for C₂₁H₃₄N₆O₂: C, 62.66; H, 8.51; N, 20.88. Found: C, 62.55; H, 8.88; N, 20.86.

### Example 10

N-[3-(4-Amino-2-hydroxy-6-pentyl-1H-imidazo[4,5-c]pyridin-1-yl)propyl]acetamide

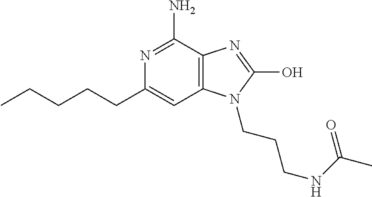

Acetyl chloride (0.05 g, 0.60 mmol) was added dropwise to a suspension of 4-amino-1-(3-aminopropyl)-6-pentyl-1H-imidazo[4,5-c]pyridin-2-ol (0.15 g, 0.54 mmol) in triethylamine (0.19 mL, 1.5 mmol) and dichloromethane (3 mL). The reaction mixture was stirred at ambient temperature for 2 hours and then partitioned between dichloromethane (50 mL) and saturated aqueous ammonium chloride (50 mL). The organic layer was dried over sodium sulfate, filtered, and then concentrated under reduced pressure to provide crude product as an oil. This material was purified by automated flash chromatography (25+M cartridge, eluting with 0% to 15% methanol in dichloromethane) followed by recrystallization from a mixture of acetonitrile (15 mL) and ethanol (3 mL) to provide 0.1 g of N-[3-(4-amino-2-hydroxy-6-pentyl-1H-imidazo[4,5-e]pyridin-1-yl)propyl]acetamide as a crystalline solid, mp 189.0-191.0° C. Anal. Calcd for C₁₆H₂₅N₅O₂: C, 60.17; H, 7.89; N, 21.93. Found: C, 59.96; H, 7.95; N, 21.98.

### Example 11

### 4-Amino-6-pentyl-1-(piperidin-4-ylmethyl)-1H-imidazo[4,5-c]pyridin-2-ol

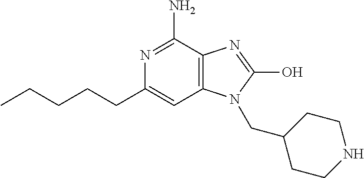

### Part A

A solution of 4-(aminomethyl)piperidine (10 g, 87.6 mmol) in chloroform (60 mL) was chilled in an ice water bath. A solution of di-tert-butyl dicarbonate (9.6 g, 43.8 mmol) in chloroform (37 mL) was added dropwise over a period of 30 minutes. The ice bath was removed and the reaction mixture was stirred at ambient temperature over the weekend. The reaction was quenched with water (100 mL). The organic layer was dried over sodium sulfate, filtered, and then concentrated under reduced pressure to provide 9 g of tert-butyl 4-(aminomethyl)piperidine-1-carboxylate.

### Part B

Tert-butyl 4-(aminomethyl)piperidine-1-carboxylate (2.2 g, 10.3 mmol) was added dropwise to a solution of 2,4-dichloro-3-nitro-6-pentylpyridine (3 g, 11.4 mmol) and triethylamine (1.7 g, 17.1 mmol) in DMF (57 mL). The reaction mixture was stirred at ambient temperature for 18 hours then quenched with water (200 ml) and extracted with ethyl acetate (2×50 mL). The organics were combined, washed with brine (50 mL), dried over sodium sulfate, filtered, and then concentrated under reduced pressure to provide crude product as an oil. This material was purified by automated flash chromatography (40+M cartridge, eluting with 0% to 10% ethyl acetate in hexanes) to provide 2.9 g of tert-butyl 4-{[(2-chloro-3-nitro-6-pentylpyridin-4-yl)amino]methyl}piperidine-1-carboxylate.

### Part C

A solution of the material from Part B (2.9 g, 6.58 mmol), N,N-bis(4-methoxybenzyl)amine (2.5 g, 9.87 mmol), and triethylamine (1 g, 9.87 mmol) in toluene (66 mL) was heated at reflux for 22 hours and then concentrated under reduced pressure to provide about 4.4 g of tert-butyl 4-[({2-[bis(4-methoxybenzyl)amino]-3-nitro-6-pentylpyridin-4-yl}amino)methyl]piperidine-1-carboxylate.

### Part D

Solid sodium borohydride (0.2 g, 5 mmol) was added in a single portion to a solution of nickel(II) chloride hexahydrate (0.78 g, 3.3 mmol) in methanol (50 mL) and the resulting suspension was stirred for 15 minutes. A solution of the material from Part C (about 6.6 mmol) in a mixture of dichloromethane (24 mL) and methanol (60 mL) was added in a single portion to the suspension. Sodium borohydride (0.25 g, 6.6 mmol) was added. After 30 minutes more sodium borohydride (0.2 g, 5 mmol) was added. Small portions of sodium borohydride were added until analysis by thin layer chromatography indicated that all of the starting material had been consumed. The reaction mixture was filtered through a layer of CELITE filter agent and the filter cake was washed with dichloromethane until the wash was clear. The filtrate was concentrated under reduced pressure. The residue was triturated with dichloromethane then filtered through a layer of CELITE filter agent. The filtrate was concentrated under reduced pressure to provide about 4.2 g of tert-butyl 4-[({3-amino-2-[bis(4-methoxybenzyl)amino]-6-pentylpyridin-4-yl}amino)methyl]piperidine-1-carboxylate as a green oil.

### Part E

A solution of the material from Part D (about 6.6 mmol) and carbonyldiimdazole (1.6 g, 9.9 mmol) in THF (33 mL) was heated at reflux for 1 hour, cooled to ambient temperature, and then concentrated under reduced pressure to provide crude product. This material was purified by automated flash chromatography (40+M cartridge, eluting with 60% to 100% ethyl acetate in hexanes) to provide 4 g of tert-butyl 4-({4-[bis(4-methoxybenzyl)amino]-2-hydroxy-6-pentyl-1H-imidazo[4,5-e]pyridin-1-yl}methyl)piperidine-1-carboxylate as a solid.

### Part F

A solution of the material from Part E in trifluoroacetic acid (15 mL) was stirred at ambient temperature for 18 hours. Water (20 mL) was added and the pH was adjusted to about 14 with 50% sodium hydroxide. The mixture was neutralized (pH 7) with 1M hydrochloric acid. The resulting suspension was stirred for 1 hour. The solid was isolated by filtration, washed with water, and dried. This material was purified by automated flash chromatography (40+M cartridge, eluting with 50% to 100% CMA in chloroform) followed by recrystallization from ethanol (22 mL) to provide 1.2 g of 4-amino-6-pentyl-1-(piperidin-4-ylmethyl)-1H-imidazo[4,5-c]pyridin-2-ol as a crystalline solid, mp 246.0-249.0° C. Anal. Calcd for C₁₇H₂₇N₅O: C, 64.32; H, 8.57; N, 22.06. Found: C, 64.10; H, 8.62; N, 21.87.

### Example 12

### 4-Amino-1-{[1-(methylsulfonyl)piperidin-4-yl]methyl}-6-pentyl-1H-imidazo[4,5-c]pyridin-2-ol

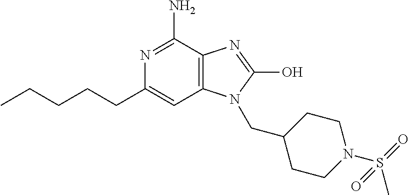

Solid methanesulfonic anhydride (0.31 g, 1.78 mmol) was added in a single portion to a suspension of 4-amino-6-pentyl-1-(piperidin-4-ylmethyl)-1H-imidazo[4,5-e]pyridin-2-ol (0.47 g, 1.48 mmol) in dichloromethane (6 mL). The reaction mixture was stirred at ambient temperature for 2 hours. More methanesulfonic anhydride (0.05 g) was added. The reaction mixture was stirred for an additional 30 minutes and then quenched with saturated aqueous sodium carbonate (20 mL) and allowed to stir overnight. A solid was isolated by filtration, washed with water, and then recrystallized from ethanol to provide 0.4 g of 4-amino-1-{[1-(methylsulfonyl)piperidin-4-yl]methyl}-6-pentyl-1H-imidazo[4,5-c]pyridin-2-ol as a solid, mp 228.0-231.0° C. Anal. Calcd for C₁₈H₂₉N₅O₃S: C, 54.66; H, 7.39; N, 17.71. Found: C, 54.45; H, 7.38; N, 17.65.

### Example 13

### 1-[(1-Acetylpiperidin-4-yl)methyl]-4-amino-6-pentyl-1H-imidazo[4,5-c]pyridin-2-ol

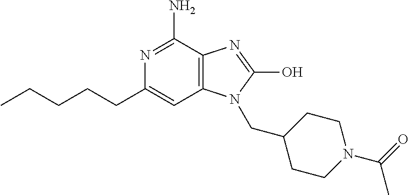

Acetyl chloride (0.15 g, 1.91 mmol) was added to a suspension of 4-amino-6-pentyl-1-(piperidin-4-ylmethyl)-1H-imidazo[4,5-c]pyridin-2-ol (0.55 g, 1.73 mmol) and triethylamine (0.44 g, 4.33 mmol) in dichloromethane (9 mL). The reaction mixture was stirred at ambient temperature for 2 hours. The reaction mixture was loaded directly onto a silica cartridge and purified by automated flash chromatography (25+M cartridge, eluting with 0% to 15% methanol in dichloromethane) to provide product as a white solid. This material was recrystallized from acetonitrile to provide 0.23 1-[(1-acetylpiperidin-4-yl)methyl]-4-amino-6-pentyl-1H-imidazo[4,5-c]pyridin-2-ol as a crystalline solid, mp 228.0-231.0° C. Anal. Calcd for C₁₉H₂₉N₅O₂: C, 63.48; H, 8.13; N, 19.48. Found: C, 63.22; H, 8.21; N, 19.33.

### Example 14

### 4-Amino-1-{[1-(ethylsulfonyl)piperidin-4-yl]methyl}-6-pentyl-1H-imidazo[4,5-c]pyridin-2-ol

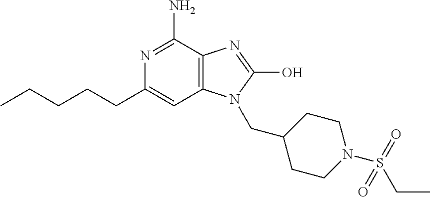

Ethanesulfonyl chloride (0.15 g, 1.13 mmol) was added to a suspension of 4-amino-6-pentyl-1-(piperidin-4-ylmethyl)-1H-imidazo[4,5-c]pyridin-2-ol (0.3 g, 0.94 mmol) and triethylamine (0.24 g, 2.36 mmol) in dichloromethane (5 mL). The reaction mixture was stirred at ambient temperature for 2 hours. The reaction mixture was quenched with methanol, which brought all of the solids into solution. The solution was loaded directly onto a silica cartridge and purified by automated flash chromatography (25+M cartridge, eluting with 0% to 10% methanol in dichloromethane) to provide 0.13 g of product as a white solid. This material was recrystallized from ethanol (7 mL) to provide 0.1 g 4-amino-1-{[1-(ethylsulfonyl)piperidin-4-yl]methyl}-6-pentyl-1H-imidazo[4,5-c]pyridin-2-ol as a crystalline solid, mp 222.0-225.0° C. Anal. Calcd for C₁₉H_{3I}N₅O₃S: C, 55.72; H, 7.63; N, 17.10. Found: C, 55.91; H, 7.53; N, 17.04.

### Example 15

### 4-Amino-1-{[1-(cyclopropylcarbonyl)piperidin-4-yl]methyl}-6-pentyl-1H-imidazo[4,5-c]pyridin-2-ol

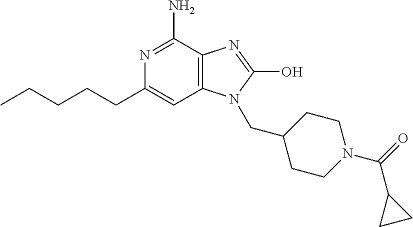

Cyclopropanecarbonyl chloride (0.12 g, 1.13 mmol) was added dropwise to a suspension of 4-amino-6-pentyl-1-(piperidin-4-ylmethyl)-1H-imidazo[4,5-c]pyridin-2-ol (0.3 g, 0.94 mmol) and triethylamine (0.24 g, 2.36 mmol) in dichloromethane (5 mL). The reaction mixture was stirred at ambient temperature for 1 hour. The reaction mixture was quenched with methanol, which brought all of the solids into solution. The solution was loaded directly onto a silica cartridge and purified by automated flash chromatography (25+M cartridge, eluting with 0% to 10% methanol in dichloromethane) to provide 0.13 g of product as a white solid. This material was recrystallized from ethanol (7 mL) to provide 0.2 g of 4-amino-1-{[1-(cyclopropylcarbonyl)piperidin-4-yl]methyl}-6-pentyl-1H-imidazo[4,5-c]pyridin-2-ol as a crystalline solid, mp 205.0-207.0° C. Anal. Calcd for C₂₁H₃₁N₅O₂: C, 65.43; H, 8.11; N, 18.17. Found: C, 65.30; H, 8.03; N, 18.22.

### Example 16

### 4-Amino-1-[4-(methylsulfonyl)butyl]-6-pentyl-1H-imidazo[4,5-c]pyridin-2-ol

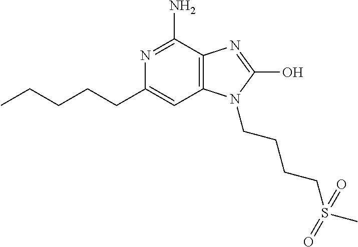

### Part A

4-(Methylsulfonyl)butan-1-amine (1.9 g of 50% pure material) was added to a solution of 2,4-dichloro-3-nitro-6-pentylpyridine (1.5 g, 5.70 mmol) and triethylamine (1.4 g, 14.2 mmol) in DMF (29 mL). The reaction mixture was stirred at ambient temperature for 18 hours and then partitioned between ethyl acetate (50 mL) and water (200 mL). The organic layer was washed with brine (50 mL), dried over sodium sulfate, filtered, and then concentrated under reduced pressure to provide crude product as a bright yellow oil. This material was purified by automated flash chromatography (40+M cartridge, eluting with 30% to 80% ethyl acetate in hexanes) to provide 1.3 g of 2-chloro-N44-(methylsulfonyl)butyl]-3-nitro-6-pentylpyridin-4-amine as a yellow oil.

### Part B

A solution of the material from Part A (1.3 g, 3.44 mmol), N,N-bis(4-methoxybenzyl)amine (1.3 g, 5.16 mmol), and triethylamine (0.5 g, 5.16 mmol) in toluene (34 mL) was heated at reflux for 22 hours and then concentrated under reduced pressure to provide about 2.1 g of N²,N²-bis(4-methoxybenzyl)-N⁴-[4-(methylsulfonyl)butyl]-3-nitro-6-pentylpyridine-2,4-diamine.

### Part C

Solid sodium borohydride (0.1 g, 2.6 mmol) was added in a single portion to a solution of nickel(II) chloride hexahydrate (0.41 g, 1.7 mmol) in methanol (30 mL) and the resulting suspension was stirred for 15 minutes. A solution of the material from Part B (about 3.4 mmol) in a mixture of dichloromethane (12 mL) and methanol (27 mL) was added in a single portion to the suspension. Sodium borohydride (0.13 g, 3.3 mmol) was added. After 30 minutes more sodium borohydride (0.2 g, 5 mmol) was added. Small portions of sodium borohydride were added until analysis by thin layer chromatography indicated that all of the starting material had been consumed. The reaction mixture was filtered through a layer of CELITE filter agent and the filter cake was washed with dichloromethane until the wash was clear. The filtrate was concentrated under reduced pressure. The residue was triturated with dichloromethane then filtered through a layer of CELITE filter agent. The filtrate was concentrated under reduced pressure to provide about 2 g of N²,N²-bis(4-methoxybenzyl)-N⁴-[4-(methylsulfonyl)butyl]-6-pentylpyridine-2,3,4-triamine as a green oil.

### Part D

A solution of the material from Part C (about 3.4 mmol) and carbonyldiimdazole (0.84 g, 5.2 mmol) in THF (17 mL) was heated at reflux for 1 hour, cooled to ambient temperature, and then concentrated under reduced pressure to provide crude product. This material was purified by automated flash chromatography (40+M cartridge, eluting with 0% to 5% methanol in dichloromethane) to provide an oil. The oil was purified by automated flash chromatography (40+M cartridge, eluting with 70% to 100% ethyl acetate in hexanes) to provide 1.6 g of 4-[bis(4-methoxybenzyl)amino]-1-[4-(methylsulfonyl)butyl]-6-pentyl-1H-imidazo[4,5-c]pyridin-2-ol as a white solid.

### Part E

A solution of the material from Part D in trifluoroacetic acid (7 mL) was stirred at ambient temperature for 2 hours. Water (20 mL) was added and the pH was adjusted to about 12 with 50% sodium hydroxide. The resulting suspension was stirred for 1 hour. The solid was isolated by filtration and washed with water to provide about 1 g of crude product as a solid. This material was purified by automated flash chromatography (40+M cartridge, eluting with 0% to 10% methanol in dichloromethane) followed by recrystallization from acetonitrile to provide 0.56 g of 4-amino-1-[4-(methylsulfonyl)butyl]-6-pentyl-1H-imidazo[4,5-c]pyridin-2-ol as a crystalline solid, mp 188.0-189.0° C. Anal. Calcd for C₁₆H₂₆N₄O₃S: C, 54.21; H, 7.39; N, 15.81. Found: C, 54.08; H, 7.51; N, 15.77.

### Example 17

### 4-Amino-142-(methylsulfonyl)ethyl)-6-pentyl-1H-imidazo[4,5-c]pyridin-2-ol

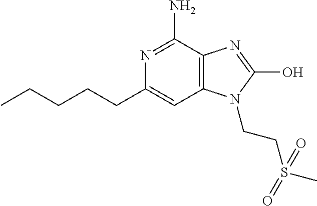

4-Amino-1-[2-(methylsulfonyl)ethyl]-6-pentyl-1H-imidazo[4,5-c]pyridin-2-ol was prepared according to the methods described in Parts A through E of Example 16 using 2-(methylsulfonyl)ethan-1-amine in lieu of 4-(methylsulfonyl)butan-1-amine in Part A. The crude product was purified by automated flash chromatography (40+M cartridge, eluting with 0% to 15% methanol in dichloromethane) followed by recrystallization from acetonitrile to provide 0.65 g of 4-amino-1-[2-(methylsulfonyl)ethyl]-6-pentyl-1H-imidazo[4,5-c]pyridin-2-ol as a crystalline solid, mp 223.0-225.0° C. Anal. Calcd for C₁₄H₂₂N₄O₃S: C, 51.51; H, 6.79; N, 17.16. Found: C, 51.80; H, 6.95; N, 17.20.

### Example 18

### 4-Amino-6-(2-ethoxyethyl)-1-[4-(methylsulfonyl)butyl]-1H-imidazo[4,5-c]pyridin-2-ol

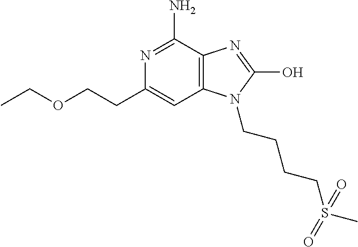

### Part A

To a solution of 3-ethoxypropionic acid (35.0 g, 296 mmol) and 1-(methylsulfonyl)benzotriazole (58.3 g, 296 mmol) in THF (400 mL) was added triethylamine (57.7 mL, 414 mmol). The resultant solution was heated to reflux overnight under a nitrogen atmosphere. The following morning, solvents were removed via rotary evaporation, and the residue was partitioned between CH₂Cl₂ and aqueous 1N HCl. The aqueous phase was extracted with CH₂Cl₂ (2×). The combined organic layers were washed with brine and dried over MgSO₄, then filtered through a silica gel plug, eluting with 1 L of 2% MeOH/CH₂Cl₂. Removal of the solvents by rotary evaporation afforded about 65 g of 1-(3-ethoxypropanoyl)-1H-1,2,3-benzotriazole as a clear, pale yellow oil that solidified upon standing. Analysis by ¹H NMR revealed product of sufficient purity to carry forward without additional purification.

### Part B

A 1 L round-bottomed flask was charged with sodium hydride (11.7 g of a 60% dispersion in oil, 293 mmol). The sodium hydride was washed with hexanes (2×); then THF (300 mL) was added to the flask. A solution of ethyl acetoacetate (34.6 g, 266 mmol) in THF (100 mL) was then added dropwise via addition funnel under a nitrogen atmosphere. A thick white precipitate formed at this point. After stirring for 1 hour, a solution of 1-(3-ethoxypropanoyl)-1H-1,2,3-benzotriazole (58.3 g, 266 mmol) in THF (100 mL) was added via addition funnel. The solution became homogeneous, then eventually a cloudy yellow mixture. This mixture was allowed to stir at room temperature under a nitrogen atmosphere overnight. The following morning, a solution of ammonium chloride (47.0 g, 878 mmol) and ammonium hydroxide (11.2 mL) in de-ionized water (50 mL) was added, and the resultant solution was heated to reflux for 2 hours. The volatile solvents were then removed by rotary evaporation, and the remaining aqueous layer was adjusted to pH 4-5 by addition of aqueous 1N HCl. The aqueous layer was then extracted with EtOAc (4×200 mL), and the combined organic layers were washed with brine, dried over MgSO₄, filtered, and concentrated to a yellow oil. This material was purified by suction filter chromatography on silica gel, eluting with 3/1 hexane/EtOAc, to afford 40.2 g (80% yield) of ethyl 5-ethoxy-3-oxopentanoate as a yellow oil. Analysis by ¹H NMR revealed material of sufficient purity to carry forward.

### Part C

To a solution of ethyl 5-ethoxy-3-oxopentanoate (40.2 g, 214 mmol) in methanol (80 mL) was added ammonium acetate (82.3 g, 1.07 mol). The resultant solution was allowed to stir at room temperature for 72 hours. The methanol was then removed by rotary evaporation, and chloroform was added to the residue. The white precipitate that formed was removed via filtration through a flitted glass funnel, and the filtrate was washed with water (2×) and brine (1×), then dried over MgSO₄, filtered, and concentrated to afford 41 g of ethyl 3-amino-5-ethoxypent-2-enoate as a yellow oil. Analysis of this material by ¹H NMR revealed it to be clean product, which was carried on without further purification.

### Part D

A solution of ethyl 3-amino-5-ethoxypent-2-enoate (40.1 g, 214 mmol) and pyridine (20.3 g, 257 mmol) in THF (400 mL) was cooled in an ice bath, and a solution of methyl malonyl chloride (32.2 g, 236 mmol) in THF (100 mL) was added dropwise via addition funnel under a nitrogen atmosphere. Upon complete addition, the resultant mixture was warmed to room temperature and allowed to stir overnight under a nitrogen atmosphere. The following morning, the mixture was diluted with water and extracted with EtOAc (3×200 mL). The combined organic layers were washed with brine, dried over MgSO₄, filtered, and concentrated to afford about 62 g of ethyl 5-ethoxy-3-[(3-methoxy-3-oxopropanoyl)amino]pent-2-enoate as a yellow oil. Analysis of this material by ¹H NMR revealed the desired product, along with unidentified impurities. The material was carried on without further purification.

### Part E

A 1 L round-bottomed flask was charged with sodium hydride (17.1 g of a 60% dispersion in oil, 428 mmol). The sodium hydride was washed with hexanes (2×); then THF (400 mL) was added to the flask. A solution of ethyl 5-ethoxy-3-[(3-methoxy-3-oxopropanoyl)amino]pent-2-enoate (61.5 g, 214 mmol) in THF (150 mL) was then added dropwise via addition funnel under a nitrogen atmosphere. Hydrogen evolution was apparent, after which time the mixture became a thick gel. Additional THF (100 mL) was added, and the reaction mixture was heated to reflux for 4 hours under a nitrogen atmosphere. The reaction was then cooled to room temperature and quenched by careful addition of methanol. The mixture was then diluted with water, and the pH was adjusted to about 4 by addition of 1N aqueous HCl. This mixture was extracted with dichloromethane (4×200 mL), and the combined organic layers were washed with brine, dried over MgSO₄, filtered, and concentrated to afford a yellow solid. This material was triturated with 1/1 hexane/EtOAc to afford 23.4 g (43% yield) of ethyl 2-(2-ethoxyethyl)-4-hydroxy-6-oxo-1,6-dihydropyridine-3-carboxylate as a light yellow solid. Purity was established by ¹H NMR.

### Part F

The material from Part E (23.4 g, 91.7 mmol) was dissolved in 70 mL of aqueous 3N HCl, and the resultant solution was heated to reflux for 24 hours. Upon cooling to room temperature, the pH of the solution was adjusted to 7 by addition of ammonium hydroxide. The water was removed by rotary evaporation, and methanol was added to the residue. The mixture was filtered through a layer of CELITE filter agent, and the solvents were removed by rotary evaporation. The residue was adsorbed onto silica gel and placed on top of a short silica gel plug. The desired product was flushed through this column, ramping the eluent from 10-30% MeOH in CH₂Cl₂. This provided 2.70 g (16% yield) of 6-(2-ethoxyethyl)pyridine-2,4-diol as a pale yellow solid. Purity was established by ¹H NMR.

### Part G

The material from Part F (2.70 g, 14.7 mmol) was dissolved in acetic acid (10 mL), and nitric acid (1.4 mL, 22.1 mmol) was added via syringe. The resultant dark solution was heated in an 85° C. oil bath for 2 hours, during which time the color became a light yellow-green. After cooling to room temperature, toluene (15 mL) was added to the solution, and solvents were removed via rotary evaporation. The residue was dissolved in MeOH, and the pH was adjusted to 7-8 by addition of ammonium hydroxide. Silica gel was added to the solution, and solvents were removed by rotary evaporation. The silica gel containing the adsorbed product was loaded onto a silica gel column, and the product was eluted with a solvent system ramped from 3/1 to 1/1 CH₂Cl₂/MeOH. This afforded 1.93 g (58% yield) of 6-(2-ethoxyethyl)-3-nitropyridine-2,4-diol as a yellow solid. Purity was established by LC-MS (229=M+H) and ¹H NMR.

### Part H

6-(2-Ethoxyethyl)-3-nitropyridine-2,4-diol (2.42 g, 10.6 mmol) was dissolved in POCl₃ (36.0 mL, 386 mmol), and the resultant yellow solution was heated in an 80° C. oil bath. Over several hours, the solution slowly turned dark in color. The bulk of the POCl₃ was removed by rotary evaporation, and the residue was quenched by careful addition of water. The pH was adjusted to 9 by addition of Na₂CO₃, and the mixture was then extracted with CH₂Cl₂ (3×50 mL). The combined organic layers were washed with brine, dried over MgSO₄, filtered, and concentrated to a dark oil. Purification via flash chromatography on silica gel (4/1 hexane/EtOAc eluent) afforded 1.23 g (44% yield) of 2,4-dichloro-6-(2-ethoxyethyl)-3-nitropyridine as a slightly tan oil that was quite clean by ¹H NMR analysis.

### Part I

The material from Part H (1.23 g, 4.64 mmol) was dissolved in CH₂Cl₂ (100 mL), and 4-(methylsulfonyl)butan-1-amine (1.54 g, 10.2 mmol) and triethylamine (1.62 mL, 11.6 mmol) were added. The resultant solution was allowed to stir at room temperature overnight under a nitrogen atmosphere. The following morning, the solution was washed with saturated aqueous NaHCO₃ and brine, then dried over MgSO₄, filtered, and concentrated to a yellow oil. Purification via flash chromatography on silica gel (ramp eluent from 2-3% MeOH in CH₂Cl₂) afforded 1.13 g (64% yield) of 2-chloro-6-(2-ethoxyethyl)-N-[4-(methylsulfonyl)butyl]-3-nitropyridin-4-amine as a thick yellow oil that was clean by ¹H NMR analysis.

### Part J

The material from Part I (1.13 g, 2.97 mmol) was dissolved in toluene (60 mL), and triethylamine (0.62 mL, 4.5 mmol) and di-para-methoxy benzylamine (1.15 g, 4.46 mmol) were added. The resultant solution was heated at reflux overnight under a nitrogen atmosphere. The following morning, the solvents were removed by rotary evaporation, and the residue was partitioned between CH₂Cl₂ and saturated aqueous NaHCO₃. The organic phase was dried over MgSO₄, filtered, and concentrated to an orange oil. Purification via flash chromatography on silica gel (2% MeOH in CH₂Cl₂ eluent) afforded 820 mg (46% yield) of 6-(2-ethoxyethyl)-N²,N²-bis(4-methoxybenzyl)-N⁴-[4-(methylsulfonyl)butyl]-3-nitropyridine-2,4-diamine as a thick orange oil that was clean by ¹H NMR analysis.

### Part K

To a solution of the material from Part J (820 mg, 1.37 mmol) in a 2:1 CH₂Cl₂/MeOH mixture (50 mL) was added nickel(II) chloride hexahydrate (162 mg, 0.68 mmol) and NaBH₄ (93 mg, 2.5 mmol). The solution instantly became black with some frothing. After 1 hour, the reaction solution was filtered through CELITE filter agent and the filter cake was washed with additional CH₂Cl₂. The filtrate was then washed with saturated aqueous NaHCO₃ and brine, dried over MgSO₄, filtered, and concentrated to afford 760 mg (97% yield) of 6-(2-ethoxyethyl)-N²,N²-bis(4-methoxybenzyl)-N⁴-[4-(methylsulfonyl)butyl]pyridine-2,3,4-triamine as a clear, colorless oil that ¹H NMR analysis showed to be of sufficient purity to carry forward without further purification.

### Part L

To a solution of the material from Part K (760 mg, 1.33 mmol) in THF (50 mL) was added N,N′-carbonyl diimidazole (324 mg, 2.00 mmol). The resultant dark green solution was heated at reflux overnight under a nitrogen atmosphere. The following morning, solvents were removed by rotary evaporation, and the residue was purified via flash chromatography on silica gel (2% MeOH in CH₂Cl₂ eluent) to provide 720 mg (91% yield) of 4-[bis(4-methoxybenzyl)amino]-6-(2-ethoxyethyl)-1-[4-(methylsulfonyl)butyl]-1H-imidazo[4,5-c]pyridin-2-ol as a thick yellow oil that solidified upon standing. ¹H NMR analysis indicated very clean product.

### Part M

The material from Part L (720 mg, 1.21 mmol) was dissolved in TFA (15 mL), and the resultant deep violet solution was allowed to stir at room temperature overnight. The following morning, the TFA was removed via rotary evaporation, and the residue was diluted with de-ionized water. The pH was then adjusted to 8-9 by addition of Na₂CO₃, and the solution was extracted with CH₂Cl₂ (2×50 mL) and a 3:1 CH₂Cl₂/MeOH mixture (60 mL). The combined organic layers were washed with brine, dried over MgSO₄, filtered, and concentrated to a tan solid. Purification via flash column chromatography on silica gel (ramp eluent from 4-10% MeOH in CH₂Cl₂) afforded 250 mg (58% yield) of 4-amino-6-(2-ethoxyethyl)-1-[4-(methylsulfonyl)butyl]-1H-imidazo[4,5-c]pyridin-2-ol as a white solid, mp 203-206° C. ¹H NMR (300 MHz, d₆-DMSO) δ 10.2 (s, 1H), 6.50 (s, 1H), 5.61 (s, 2H), 3.73 (m, 2H), 3.63 (t, J=7.2 Hz, 2H), 3.42 (q, J=7.0 Hz, 21-1), 3.33 (s, 3H), 3.16 (m, 2H), 2.93 (s, 3H), 2.74 (t, J=7.2 Hz, 2H), 1.71 (m, 4H), 1.09 (t, J=7.0 Hz, 3H). MS m/z 357 (M+H⁺); Anal. calcd for C₁₅H₂₄N₄O₄S: C, 50.54; H, 6.79; N, 15.72. Found: C, 50.47; H, 6.68; N, 15.57.

### Example 19

### 4-Amino-1-{[3-(4-fluorophenypisoxazol-5-yl]methyl}-6,7-dimethyl-1H-imidazo[4,5-c]pyridin-2-ol

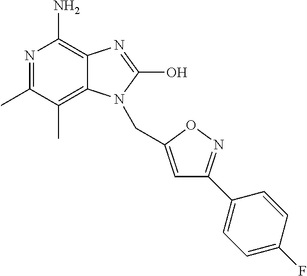

### Part A

To a solution of 2-chloro-5,6-dimethyl-3-nitro-N-prop-2-ynylpyridin-4-amine (10.0 g, 41.7 mmol), see International Publication No. WO2006/065280 (Moser et al.) Example 18, in toluene (200 mL) was added di-para-methoxy benzylamine (16.1 g, 62.6 mmol) and triethylamine (8.7 mL, 62.6 mmol). The resultant solution was heated to reflux for 58 hours. Upon cooling, solvents were removed by rotary evaporation, and the residue was partitioned between CH₂Cl₂ and saturated aqueous NaHCO₃. The aqueous phase was extracted with CH₂Cl₂ (3×100 mL), and the combined organic layers were washed with brine, dried over MgSO₄, filtered, and concentrated to afford a waxy red solid. Purification via suction filter chromatography on silica gel (3/1 hexane/EtOAc eluent) afforded 18.5 g (96% yield) of N²,N²-bis(4-methoxybenzyl)-5,6-dimethyl-3-nitro-N⁴-prop-2-ynylpyridine-2,4-diamine as a bright orange solid that was quite pure by ¹H NMR analysis.

### Part B

N²,N²-bis(4-methoxybenzyl)-5,6-dimethyl-3-nitro-N⁴-prop-2-ynylpyridine-2,4-diamine (11.5 g, 25.0 mmol) was dissolved in a 1:1 EtOH/CH₃CN mixture (300 mL), and a solution of sodium dithionite (21.7 g, 125 mmol) in de-ionized water (100 mL) was added. A precipitate formed immediately, and the resultant mixture was allowed to stir at room temperature for 45 min. The precipitate was then removed by filtration through a pad of CELITE filter agent and the filter cake was washed with CH₂Cl₂. The volatile solvents were removed by rotary evaporation, and the remaining residue was partitioned between saturated aqueous NaHCO₃ and EtOAc. The aqueous phase was extracted with additional EtOAc (3×100 mL), and the combined organic layers were washed with brine, dried over MgSO₄, filtered, and concentrated to afford 7.13 g (66% yield) of N²,N²-bis(4-methoxybenzyl)-5,6-dimethyl-N⁴-prop-2-ynylpyridine-2,3,4-triamine as a yellow solid. Analysis by ¹H NMR revealed product of sufficient purity to carry forward without additional purification.

### Step C

To a solution of the material from Part B (7.13 g, 16.6 mmol) in THF (100 mL) was added N,N′-carbonyl diimidazole (4.03 g, 24.8 mmol). The resultant solution was heated at reflux for 24 hours under a nitrogen atmosphere. Upon cooling, the solvents were removed by rotary evaporation, and the residue was partitioned between CH₂Cl₂ and aqueous 1N HCl. The organic phase was subsequently washed with brine, dried over MgSO₄, filtered, and concentrated to an orange solid. Initial purification via suction filter chromatography on silica gel (1/1 EtOAc/CH₂Cl₂ eluent) removed non-polar impurities; subsequent crystallization from EtOAc provided 1.5 g of 4-[bis(4-methoxybenzyl)amino]-6,7-dimethyl-1-prop-2-ynyl-1H-imidazo[4,5-c]pyridin-2-ol as a white crystalline solid, along with an additional 5 g of material which required further purification.

### Part D

A solution of 4-fluorobenzaldehyde oxime (0.97 g, 7.0 mmol), see International Publication No. WO2006/065280 (Moser et al.) Example 11, in DMF (14 mL) was chilled in an ice/water bath. N-Chlorosuccinimide (0.93 g, 7.0 mmol) was added in a single portion. The ice bath was removed; the solution was stirred at ambient temperature for 2 hours and then partitioned between ethyl acetate (50 mL) and water (50 mL). The layers were separated and the aqueous layer was extracted with ethyl acetate (2×50 mL). The combined organics were washed with brine (50 mL), dried over sodium sulfate, filtered, and then concentrated under reduced pressure to provide about 1.2 g of 4-fluoro-N-hydroxybenzenecarboximidoyl chloride as a light yellow solid.

### Part E

A suspension of the material from part D (about 7 mmol) in chloroform (23 mL) was cooled in an ice/water bath. Solid 4-[bis(4-methoxybenzyl)amino]-6,7-dimethyl-1-prop-2-ynyl-1H-imidazo[4,5-c]pyridin-2-ol (1.3 g, 2.8 mmol) was added, followed by triethylamine (1.5 mL, 10.5 mmol). The ice bath was removed and the suspension was stirred at ambient temperature for 18 hours by which time a solution had been obtained. The reaction was quenched with saturated aqueous ammonium chloride (30 mL) The organic layer was dried over sodium sulfate, filtered, and then concentrated under reduced pressure to provide an oil. This material was purified by automated flash chromatography (40+M cartridge, eluting with 20 to 60% ethyl acetate in hexanes) to provide 0.84 g of 4-[bis(4-methoxybenzyl)amino]-1-{[3-(4-fluorophenyl)isoxazol-5-yl]methyl}-6,7-dimethyl-1H-imidazo[4,5-c]pyridin-2-ol.

### Part F

A solution of the material from Part E in trifluoroacetic acid (4 mL) was stirred at ambient temperature for 2 hours. The reaction was quenched with water (20 mL) and a white precipitate formed. The pH of the suspension was adjusted to about 13 with 50% aqueous sodium hydroxide. The mixture was stirred for 1 hour. The solid was isolated by filtration, washed with water, and then purified by automated flash chromatography (40+M cartridge, eluting with 0 to 15% methanol in dichloromethane) to provide a solid. The solid was combined with ethanol (60 mL) and heated to reflux. The mixture was allowed to cool. A solid was isolated by filtration, washed with ethanol, and then dried under vacuum at 65° C. to provide 0.3 g of 4-amino-1-{[3-(4-fluorophenyl)isoxazol-5-yl]methyl}-6,7-dimethyl-1H-imidazo[4,5-c]pyridin-2-ol as a crystalline solid, mp 250.0° C. Anal. Calcd for C₁₈H₁₆FN₅O₂: C, 61.18; H, 4.56; N, 19.82. Found: C, 60.99; H, 4.62; N, 19.55.

### Example 20

### 4-Amino-6,7-dimethyl-1-[(3-methylisoxazol-5-yl)methyl]-1H-imidazo[4,5-c]pyridin-2-ol

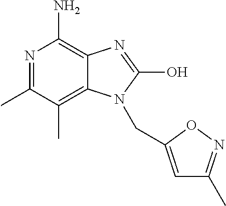

### Part A

A solution of acetaldoxime (0.43 g, 7.4 mmol) and N-chlorosuccinimide (0.98 g, 7.4 mmol) in DMF (22 mL) was heated at 50° C. for 2 hours. The reaction mixture was allowed to cool to ambient temperature; then it was quenched with water (150 mL) and extracted with ethyl acetate (2×50 mL). The combined organics were washed with brine (50 mL), dried over sodium sulfate, filtered, and then concentrated under reduced pressure to provide 0.45 g of N-hydroxyethanimidoyl chloride as a clear oil.

### Part B

A solution of the material from Part A (4.8 mmol) in dichloromethane (25 mL) was cooled in an ice/water bath. Solid 4-[bis(4-methoxybenzyl)amino]-6,7-dimethyl-1-prop-2-ynyl-1H-imidazo[4,5-c]pyridin-2-ol (1.8 g, 3.9 mmol) was added, followed by triethylamine (0.82 mL, 5.9 mmol). The ice bath was removed. The reaction solution was stirred at ambient temperature for 68 hours and then concentrated under reduced pressure to provide crude product as an oil. This material was purified by automated flash chromatography (40+M cartridge, eluting with 0% to 5% methanol in dichloromethane) to provide 0.58 g of 4-[bis(4-methoxybenzyl)amino]-6,7-dimethyl-1-[(3-methylisoxazol-5-yl)methyl]-1H-imidazo[4,5-c]pyridin-2-ol as a white solid.

### Part C

A solution of the material from Part B in trifluoroacetic acid (3 mL) was stirred at ambient temperature for 18 hours. The reaction was quenched with water (20 mL). The pH was adjusted to about 14 with 50% aqueous sodium hydroxide and then the mixture was neutralized (pH 7) with 1M aqueous hydrochloric acid. The resulting suspension was stirred for 1 hour. The solid was isolated by filtration, washed with water, and dried. This material was purified by automated flash chromatography (25+M cartridge, eluting with 50% to 100% CMA in chloroform) followed by recrystallization from methanol to provide 0.13 g of 4-amino-6,7-dimethyl-1-[(3-methylisoxazol-5-yl)methyl]-1H-imidazo[4,5-c]pyridin-2-ol as a crystalline solid, mp>300° C. Anal. Calcd for C₁₃H₁₅N₅O₂: C, 57.13; H, 5.53; N, 25.63. Found: C, 57.15; H, 5.75; N, 25.81.

### Example 21

### 4-Amino-6-pentyl-1-[(1S)-1-phenylethyl]-1H-imidazo[4,5-c]pyridin-2-ol

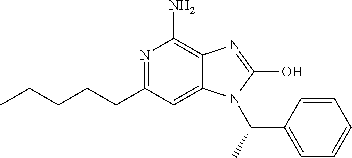

### Part A

A solution of 2,4-dichloro-3-nitro-6-pentylpyridine (1.11 g, 4.22 mmol) dissolved in 10 mL of N,N-dimethylformamide was treated with triethylamine (1.17 mL, 8.44 mmol) and (D)-(+)-phenethylamine (536 mL, 4.22 mmol). After stirring for 6 hours at ambient temperature, the reaction mixture was heated to 40° C. for 1 hour and then concentrated under reduced pressure. The residue was dissolved in 50 mL of ethyl acetate and washed successively with H₂O (3×) and brine. The organic portion was dried over Na₂SO₄, filtered, and concentrated to give yellow oil. Column chromatography (SiO₂, 3-15% methyl tert-butylether (MTBE)/hexanes) gave the 2-chloro-3-nitro-6-pentyl-N-[(1S)-1-phenylethyl]pyridin-4-amine (715 mg) as a yellow syrup.

### Part B

A solution of 2-chloro-3-nitro-6-pentyl-N-[(1S)-1-phenylethyl]pyridin-4-amine (715 mg, 2.05 mmol) dissolved in 20 mL of toluene was treated with triethylamine (0.57 mL, 4.1 mmol) and di-p-methoxybenzylamine (581 mg, 2.26 mmol) and the mixture was heated to reflux overnight. The reaction mixture was concentrated under reduced pressure and the residue was dissolved in 50 mL of ethyl acetate and washed successively with H₂O and brine. The organic portion was dried over Na₂SO₄, filtered, and concentrated to give brown oil. Column chromatography (SiO₂, 3-15% MTBE/hexanes) gave the N²,N²-bis(4-methoxybenzyl)-3-nitro-6-pentyl-N⁴-[(1S)-1-phenylethyl]pyridine-2,4-diamine (1.10 g) as a dark-yellow syrup.

### Part C

A stirred solution of Ni(II) chloride hexahydrate (230 mg) dissolved in 10 mL of methanol was treated with NaBH₄ (74 mg). A solution of N²,N²-bis(4-methoxybenzyl)-3-nitro-6-pentyl-N⁴-[(1S)-1-phenylethyl]pyridine-2,4-diamine (1.10 g, 1.94 mmol), dissolved in 10 mL of a 1:1 mixture of methanol/CH₂Cl₂, was then added to the stirred solution. Additional 20 mg-portions of NaBH₄ (about 8) were then added over 30 minutes until the reaction mixture turned from yellow-brown to clear. Thin-layer chromatography showed the complete consumption of starting material. The reaction mixture was then filtered though a layer of CELITE filter agent. The filter cake was rinsed with additional CH₂Cl₂ and the combined filtrates were concentrated under reduced pressure. The resulting material was then filtered through a short column of SiO₂, eluting with 5-10% methanol/CHCl₃ to give N²,N²-bis(4-methoxybenzyl)-6-pentyl-N⁴-[(1S)-1-phenylethyl]pyridine-2,3,4-triamine (979 mg) as a light brown solid.

### Part D

A solution of N²,N²-bis(4-methoxybenzyl)-6-pentyl-N⁴-[(1S)-1-phenylethyl]pyridine-2,3,4-triamine (979 mg, 1.82 mmol) dissolved in 10 mL of THF was treated with carbonyl diimidazole (590 mg, 3.64 mmol) and the mixture was heated to reflux. After 90 minutes, the reaction mixture was treated with an additional portion (200 mg) of carbonyl diimidazole and heating was continued for 1 hour. The reaction mixture was cooled and treated with 10 mL of H₂O. After stirring for 10 minutes, the reaction mixture was diluted with 30 mL of ethyl acetate. The layers were separated and the organic portion was washed successively with H₂O and brine. The organic portion was dried over Na₂SO₄, filtered, and concentrated to give a purple oil. Column chromatography (SiO₂, 0-5% methanol/CH₂Cl₂) gave the 4-[bis(4-methoxybenzyl)amino]-6-pentyl-1-[(1S)-1-phenylethyl]-1H-imidazo[4,5-c]pyridin-2-ol (977 mg) as a purple syrup.

### Part E

A solution of 4-[bis(4-methoxybenzyl)amino]-6-pentyl-1-[(1S)-1-phenylethyl]-1H-imidazo[4,5-c]pyridin-2-ol (977 mg, 1.73 mmol) dissolved in 10 mL of trifluoroacetic acid (TFA) was stirred overnight. The reaction mixture was then concentrated under reduced pressure and the resulting residue was partitioned between dilute NH₄OH and CH₂Cl₂. The layers were separated and the aqueous portion was extracted with 2 additional portions of CH₂Cl₂. The combined organic portions were dried over Na₂SO₄, filtered, and concentrated under reduced pressure. Column chromatography (SiO₂, 10-35% CMA/CHCl₃) gave the title compound, which was further purified by crystallization from 10 mL of hot acetonitrile. The crystals were isolated by filtration and dried under high vacuum to give 4-amino-6-pentyl-1-[(1S)-1-phenylethyl]-1H-imidazo[4,5-c]pyridin-2-ol (340 mg) as pinkish crystals: mp 146.7-147.7° C.; ¹H NMR (500 MHz, CDCl₃) δ 10.90 (br s, 1H), 7.22-7.39 (m, 5H), 6.02 (s, 1H), 5.74 (q, J=7.2 Hz, 1H), 4.92 (s, 2H), 2.48 (t, J=7.7 Hz, 2H), 1.89 (d, J=7.2 Hz, 1H), 1.53 (m, 2H), 1.17-1.29 (m, 4H), 0.84 (t, J=7.1 Hz, 3H); ¹³C NMR (125 MHz, CDCl₃) δ 154.9, 154.0, 142.7, 139.4, 136.0, 128.7, 127.7, 126.4, 108.4, 96.5, 51.0, 38.4, 31.5, 29.8, 22.5, 17.8, 14.0; MS m/z 325 (M+H)⁺. Anal. calcd for C₁₉H₂₄N₄O: C, 70.34; H, 7.46; N, 17.27. Found: C, 70.30; H, 7.51; N, 17.22. Optical rotation [α]=−58.6 (c=1.33 mg/mL, CH₂Cl₂).

### Example 22

### 4-Amino-6-pentyl-1-[(1R)-1-phenylethyl]-1H-imidazo[4,5-c]pyridin-2-ol

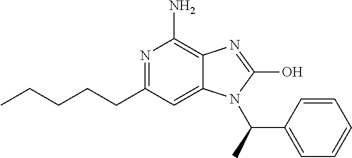

4-Amino-6-pentyl-1-[(1R)-1-phenylethyl]-1H-imidazo[4,5-c]pyridin-2-ol was prepared according to the methods described in Parts A through E of Example 21 using (L)-(−)-phenethylamine in lieu of (D)-(+)-phenethylamine in Part A. mp 147.1-148.0° C.; Anal. calcd for C₁₉H₂₄N₄O: C, 70.34; H, 7.46; N, 17.27. Found: C, 70.41; H, 7.51; N, 17.40. Optical rotation [α]=+61.5 (c=1.82 mg/mL, CH₂Cl₂).

### Example 23

### 4-amino-1-benzyl-6-butoxy-1H-imidazo[4,5-c]pyridin-2-ol

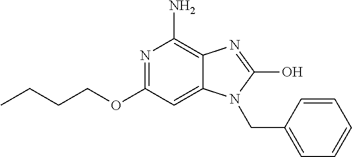

### Part A

4-Amino-2,6-dichloropyridine (1.30 g, 7.98 mmol) was carefully added to 6.5 mL of concentrated sulfuric acid. The mixture was cooled in an ice bath, and 2.6 mL of fuming nitric acid was added dropwise via pipette. The solution was warmed to room temperature, stirred for one hour, and then poured onto 26 g of crushed ice, resulting in the formation of a white precipitate. The mixture was stored at −10° C. overnight. The white precipitate was collected by filtration using a Buchner funnel, washed with ice cold water, and dried under vacuum to provide 1.66 g of 2,6-dichloro-4-nitraminopyridine, which was carried forward without additional purification.

### Part B

2,6-Dichloro-4-nitraminopyridine (1.66 g, 7.98 mmol) was added to 11 mL of concentrated sulfuric acid, and the resultant solution was heated on a steam bath for 30 minutes. After cooling to room temperature, the solution was poured onto 28 g of crushed ice, resulting in the formation of a tan precipitate. The mixture was cooled in an ice bath, and concentrated ammonium hydroxide was added until pH 7 was reached. The resultant slurry was stored at −10° C. overnight. The precipitate was collected by filtration using a Buchner funnel, washed with ice cold water, and dried under vacuum to provide 4-amino-2,6-dichloro-3-nitropyridine (1.30 g, 78% yield) as a light tan solid.

### Part C

A solution of 4-amino-2,6-dichloro-3-nitropyridine (1.08 g, 5.19 mmol) from Part B and triethylamine (1.09 mL, 7.79 mmol) in dichloromethane (20 mL) was cooled in an ice bath, and bis(4-methoxybenzyl)amine (1.34 g, 5.19 mmol) was added in one portion. The resultant solution was allowed to warm to room temperature and stirred overnight under a nitrogen atmosphere. The solvents were removed by rotary evaporation, and the residue was purified via flash chromatography (silica gel, dichloromethane eluent) to provide N²,N²-bis(4-methoxybenzyl)-6-chloro-3-nitropyridine-2,4-diamine (1.82 g, 82% yield) as a viscous yellow oil that foamed under vacuum.

### Part D

Sodium butoxide was prepared by adding sodium metal (207 mg, 9.00 mmol) to 1-butanol (7 mL). After the sodium metal had been completely consumed, the resulting solution was cooled in an ice bath, and a solution of N²,N²-bis(4-methoxybenzyl)-6-chloro-3-nitropyridine-2,4-diamine (1.29 g, 3.00 mmol) from Part C in THF (10 mL) was added dropwise via an addition funnel. The resultant solution was heated in an 85° C. oil bath for five hours. Upon cooling to room temperature, the reaction mixture was quenched by the addition of dilute aqueous HCl and extracted with dichloromethane (3×50 mL). The combined organic layers were washed with brine, dried over MgSO₄, filtered, and concentrated to a red oil. Purification via flash chromatography (silica gel, dichloromethane eluent) provided N²,N²-bis(4-methoxybenzyl)-6-butoxy-3-nitropyridine-2,4-diamine (1.14 g, 81% yield) as a viscous orange oil that foamed under vacuum.

### Part E

N²,N²-Bis(4-methoxybenzyl)-6-butoxy-3-nitropyridine-2,4-diamine (1.10 g, 2.36 mmol) from Part D was dissolved in 40 mL of a 1:1 ethanol/acetonitrile mixture, and a solution of sodium hydrosulfite, Na₂S₂O₄, (2.05 g, 11.8 mmol) in H₂O (10 mL) was added via pipette, resulting in the formation of a white precipitate. The mixture was stirred at room temperature for two hours, during which time the orange-yellow color faded away. The mixture was then filtered through a pad of CELITE filter agent, the filter cake was washed with dichloromethane, and the filtrate was concentrated under reduced pressure. The residue was diluted with ethyl acetate (150 mL), washed with saturated aqueous sodium bicarbonate (1×50 mL) and brine (1×25 mL), dried over magnesium sulfate, filtered, and concentrated to provide N²,N²-bis(4-methoxybenzyl)-6-butoxy-pyridine-2,3,4-diamine as a yellow oil. This material was carried forward without additional purification.

### Part F

To a solution of N²,N²-bis(4-methoxybenzyl)-6-butoxy-pyridine-2,3,4-diamine (1.03 g, 2.35 mmol) from Part E in THF (25 mL) was added 1,1′-carbonyldiimidazole (496 mg, 3.06 mmol). The resultant solution was heated to reflux overnight under a nitrogen atmosphere. The solvents were removed by rotary evaporation, and the residue was purified via flash chromatography (silica gel, ramp eluent from 2/1 to 1/2 hexane/ethyl acetate) to provide 6-butoxy-4-[di(4-methoxybenzyl)amino]-1,3-dihydroimidazo[4,5-c]pyridin-2-one (630 mg, 58% yield over two steps) as a light pink oil that foamed under vacuum.

### Part G

The 6-butoxy-4-[di(4-methoxybenzyl)amino]-1,3-dihydroimidazo[4,5-c]pyridin-2-one (630 mg, 1.36 mmol) from Part F was dissolved in DMF (8 mL), and solid potassium carbonate (225 mg, 1.63 mmol) was added. A solution of benzyl bromide (257 mg, 1.50 mmol) in DMF (2 mL) was then added via pipette, and the resultant solution was heated at 80° C. in an oil bath overnight under a nitrogen atmosphere. The reaction mixture was diluted with ethyl acetate (150 mL), washed with water (4×25 mL) and brine (4×25 mL), dried over magnesium sulfate, filtered, and concentrated to give an oil. Purification by flash chromatography (silica gel, ramp eluent from 2/1 to 1/2 hexane/ethyl acetate) provided 1-benzyl-6-butoxy-4-[di(4-methoxybenzyl)amino]-1H-imidazo[4,5-c]pyridin-2-ol (80 mg, 11% yield) as a tan oil.

### Part H

To the 1-benzyl-6-butoxy-4-[di(4-methoxybenzyl)amino]-1H-imidazo[4,5-c]pyridin-2-ol (80 mg, 0.15 mmol) from Part G was added TFA (5 mL), forming a deep violet solution, which was allowed to stir at room temperature overnight. The TFA was removed via rotary evaporation, and the residue was diluted with water (10 mL). Solid sodium carbonate was added to adjust the pH to about 8-9. The aqueous layer was extracted with dichloromethane (3×25 mL). The combined organic layers were dried over magnesium sulfate, filtered, and concentrated to give a tan solid. The tan solid was purified by flash chromatography (silica gel, 6% methanol in dichloromethane eluent) to provide 4-amino-1-benzyl-6-butoxy-1H-imidazo[4,5-c]pyridin-2-ol (30 mg, 66% yield) as a light tan solid, mp 205-208° C. ¹H NMR (500 MHz, CDCl₃) δ 9.41 (s, 1H), 7.47-7.24 (m, 5H), 5.99 (s, 1H), 5.22 (s, 2H), 4.08 (t, J=7.1 Hz, 2H), 3.98 (s, 2H), 1.70 (m, 2H), 1.44 (m, 2H), 0.94 (t, J=7.1 Hz, 3H). MS m/z 313 (M+H⁺).

### Exemplary Compounds

Certain exemplary compounds, including some of those described above in the Examples, have the following Formula (Ia) and an R₁ substituent shown in the following table, wherein each line of the table is matched with the Formula (Ia) to represent a specific embodiment of the invention.

Ia

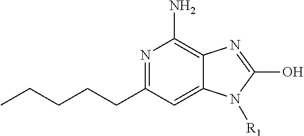

R₁

pyridin-3-ylmethyl
4-fluorobenzyl
isoxazol-5-ylmethyl
isoxazol-3-ylmethyl
[5-(4-fluorophenyl)isoxazol-3-
yl]methyl
[3-(4-fluorophenyl)isoxazol-5-
yl]methyl

Compounds of the invention have been found to modulate cytokine biosynthesis by inducing the production of interferon α, or interferon α and tumor necrosis factor α in human cells when tested using one of the methods described below.

### Cytokine Induction in Human Cells

An in vitro human blood cell system is used to assess cytokine induction. Activity is based on the measurement of interferon (α) and tumor necrosis factor (α) (IFN-α and TNF-α, respectively) secreted into culture media as described by Testerman et al. in “Cytokine Induction by the Immunomodulators Imiquimod and S-27609”, Journal of Leukocyte Biology, 58, 365-372 (September, 1995).

### Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Blood is diluted 1:1 with Dulbecco's Phosphate Buffered Saline (DPBS) or Hank's Balanced Salts Solution (HBSS). Alternately, whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete. The PBMC suspension is added to 96 well flat bottom sterile tissue culture plates containing an equal volume of RPMI complete media containing test compound.

### Compound Preparation

The compounds are solubilized in dimethyl sulfoxide (DMSO). The DMSO concentration should not exceed a final concentration of 1% for addition to the culture wells. The compounds are generally tested at concentrations ranging from 30-0.014 μM. Controls include cell samples with media only, cell samples with DMSO only (no compound), and cell samples with reference compound.

### Incubation

The solution of test compound is added at 60 μM to the first well containing RPMI complete and serial 3 fold dilutions are made in the wells. The PBMC suspension is then added to the wells in an equal volume, bringing the test compound concentrations to the desired range (usually 30-0.014 μM). The final concentration of PBMC suspension is 2×10⁶ cells/mL. The plates are covered with sterile plastic lids, mixed gently and then incubated for 18 to 24 hours at 37° C. in a 5% carbon dioxide atmosphere.

### Separation

Following incubation the plates are centrifuged for 10 minutes at 1000 rpm (approximately 200×g) at 4° C. The cell-free culture supernatant is removed and transferred to sterile polypropylene tubes. Samples are maintained at −30 to −70° C. until analysis. The samples are analyzed for IFN-α by ELISA and for TNF-α by IGEN/BioVeris Assay.

### Interferon (α) and Tumor Necrosis Factor (α) Analysis

IFN-α concentration is determined with a human multi-subtype colorimetric sandwich ELISA (Catalog Number 41105) from PBL Biomedical Laboratories, Piscataway, N.J. Results are expressed in pg/mL.

The TNF-α concentration is determined by ORIGEN M-Series Immunoassay and read on an IGEN M-8 analyzer from BioVeris Corporation, formerly known as IGEN International, Gaithersburg, Md. The immunoassay uses a human TNF-α capture and detection antibody pair (Catalog Numbers AHC3419 and AHC3712) from Biosource International, Camarillo, Calif. Results are expressed in pg/mL.

### Assay Data and Analysis

In total, the data output of the assay consists of concentration values of TNF-α and IFN-α (y-axis) as a function of compound concentration (x-axis).

Analysis of the data has two steps. First, the greater of the mean DMSO (DMSO control wells) or the experimental background (usually 20 pg/mL for IFN-α and 40 pg/mL for TNF-α) is subtracted from each reading. If any negative values result from background subtraction, the reading is reported as “*”, and is noted as not reliably detectable. In subsequent calculations and statistics, “*”, is treated as a zero. Second, all background subtracted values are multiplied by a single adjustment ratio to decrease experiment to experiment variability. The adjustment ratio is the area of the reference compound in the new experiment divided by the expected area of the reference compound based on the past 61 experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from the past 61 experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

### Cytokine Induction in Human Cells

### High Throughput Screen

The CYTOKINE INDUCTION IN HUMAN CELLS test method described above was modified as follows for high throughput screening.

### Blood Cell Preparation for Culture

Whole blood from healthy human donors is collected by venipuncture into vacutainer tubes or syringes containing EDTA. Peripheral blood mononuclear cells (PBMC) are separated from whole blood by density gradient centrifugation using HISTOPAQUE-1077 (Sigma, St. Louis, Mo.) or Ficoll-Paque Plus (Amersham Biosciences Piscataway, N.J.). Whole blood is placed in Accuspin (Sigma) or LeucoSep (Greiner Bio-One, Inc., Longwood, Fla.) centrifuge frit tubes containing density gradient medium. The PBMC layer is collected and washed twice with DPBS or HBSS and re-suspended at 4×10⁶ cells/mL in RPMI complete (2-fold the final cell density). The PBMC suspension is added to 96-well flat bottom sterile tissue culture plates.

### Compound Preparation

The compounds are solubilized in dimethyl sulfoxide (DMSO). The compounds are generally tested at concentrations ranging from 30-0.014 μM. Controls include cell samples with media only, cell samples with DMSO only (no compound), and cell samples with a reference compound 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) on each plate. The solution of test compound is added at 7.5 mM to the first well of a dosing plate and serial 3 fold dilutions are made for the 7 subsequent concentrations in DMSO. RPMI Complete media is then added to the test compound dilutions in order to reach a final compound concentration of 2-fold higher (60-0.028 μM) than the final tested concentration range.

### Incubation

Compound solution is then added to the wells containing the PBMC suspension bringing the test compound concentrations to the desired range (usually 30-0.014 μM) and the DMSO concentration to 0.4%. The final concentration of PBMC suspension is 2×10⁶ cells/mL. The plates are covered with sterile plastic lids, mixed gently and then incubated for 18 to 24 hours at 37° C. in a 5% carbon dioxide atmosphere.

### Separation

Following incubation the plates are centrifuged for 10 minutes at 1000 rpm (approximately 200 g) at 4° C. 4-plex Human Panel MSD MULTI-SPOT 96-well plates are pre-coated with the appropriate capture antibodies by MesoScale Discovery, Inc. (MSD, Gaithersburg, Md.). The cell-free culture supernatants are removed and transferred to the MSD plates. Fresh samples are typically tested, although they may be maintained at −30 to −70° C. until analysis.

### Interferon-α and Tumor Necrosis Factor-α Analysis

MSD MULTI-SPOT plates contain within each well capture antibodies for human TNF-α and human IFN-α that have been pre-coated on specific spots. Each well contains four spots: one human TNF-α capture antibody (MSD) spot, one human IFN-α capture antibody (PBL Biomedical Laboratories, Piscataway, N.J.) spot, and two inactive bovine serum albumin spots. The human TNF-α capture and detection antibody pair is from MesoScale Discovery. The human IFN-α multi-subtype antibody (PBL Biomedical Laboratories) captures all IFN-α subtypes except IFN-α F (IFNA21). Standards consist of recombinant human TNF-α (R&D Systems, Minneapolis, Minn.) and IFN-α (PBL Biomedical Laboratories). Samples and separate standards are added at the time of analysis to each MSD plate. Two human IFN-a, detection antibodies (Cat. Nos. 21112 & 21100, PBL) are used in a two to one ratio (weight:weight) to each other to determine the IFN-α concentrations. The cytokine-specific detection antibodies are labeled with the SULFO-TAG reagent (MSD). After adding the SULFO-TAG labeled detection antibodies to the wells, each well's electrochemoluminescent levels are read using MSD's SECTOR HTS READER. Results are expressed in pg/mL upon calculation with known cytokine standards.

### Assay Data and Analysis

In total, the data output of the assay consists of concentration values of TNF-α or IFN-α (y-axis) as a function of compound concentration (x-axis).

A plate-wise scaling is performed within a given experiment aimed at reducing plate-to-plate variability associated within the same experiment. First, the greater of the median DMSO (DMSO control wells) or the experimental background (usually 20 pg/mL for IFN-α and 40 pg/mL for TNF-α) is subtracted from each reading. Negative values that may result from background subtraction are set to zero. Each plate within a given experiment has a reference compound that serves as a control. This control is used to calculate a median expected area under the curve across all plates in the assay. A plate-wise scaling factor is calculated for each plate as a ratio of the area of the reference compound on the particular plate to the median expected area for the entire experiment. The data from each plate are then multiplied by the plate-wise scaling factor for all plates. Only data from plates bearing a scaling factor of between 0.5 and 2.0 (for both cytokines IFN-α, TNF-α) are reported. Data from plates with scaling factors outside the above mentioned interval are retested until they bear scaling factors inside the above mentioned interval. The above method produces a scaling of the y-values without altering the shape of the curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91). The median expected area is the median area across all plates that are part of a given experiment.

A second scaling may also be performed to reduce inter-experiment variability (across multiple experiments). All background-subtracted values are multiplied by a single adjustment ratio to decrease experiment-to-experiment variability. The adjustment ratio is the area of the reference compound in the new experiment divided by the expected area of the reference compound based on an average of previous experiments (unadjusted readings). This results in the scaling of the reading (y-axis) for the new data without changing the shape of the dose-response curve. The reference compound used is 2-[4-amino-2-ethoxymethyl-6,7,8,9-tetrahydro-α,α-dimethyl-1H-imidazo[4,5-c]quinolin-1-yl]ethanol hydrate (U.S. Pat. No. 5,352,784; Example 91) and the expected area is the sum of the median dose values from an average of previous experiments.

The minimum effective concentration is calculated based on the background-subtracted, reference-adjusted results for a given experiment and compound. The minimum effective concentration (μmolar) is the lowest of the tested compound concentrations that induces a response over a fixed cytokine concentration for the tested cytokine (usually 20 pg/mL for IFN-α and 40 pg/mL for TNF-α). The maximal response is the maximal amount of cytokine (pg/ml) produced in the dose-response.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

We claim:

1. A compound of Formula I: 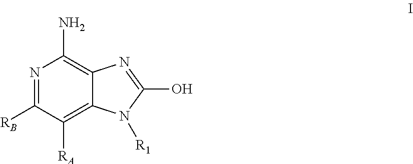
wherein:
R_{A} is selected from the group consisting of hydrogen and alkyl;
R_{B} is selected from the group consisting of alkyl, alkoxyalkylenyl, and —OR₁₁;
R₁₁ is alkyl wherein the alkyl group is unsubstituted or substituted by one substituent selected from the group consisting of alkyl; alkoxy; hydroxy; hydroxyalkyl; aryl; aryloxy; arylalkyleneoxy; heteroaryl; heteroaryloxy; heteroarylalkyleneoxy; halogen; haloalkyl; haloalkoxy; mercapto; nitro; cyano; heterocyclyl; amino; alkylamino; dialkylamino; and oxo;
R₁ is selected from the group consisting of —R₄ and —X—R₄;
X is alkylene wherein the alkylene group is optionally interrupted by one —O—; and
R₄ is selected from the group consisting of alkyl, aryl, heteroaryl, and heterocyclyl wherein the alkyl, aryl, heteroaryl, and heterocyclyl groups can be unsubstituted or substituted by one substituent selected from the group consisting of alkyl; alkoxy; hydroxyalkyl; haloalkyl; haloalkoxy; halogen; nitro; hydroxy; mercapto; cyano; aryl; aryloxy; arylalkyleneoxy; heteroaryl; heteroaryloxy; heteroarylalkyleneoxy; heterocyclyl; amino; alkylamino; dialkylamino; (dialkylamino)alkyleneoxy; and, in the case of alkyl and heterocyclyl, oxo; or a pharmaceutically acceptable salt thereof.

2. The compound or salt of claim 1 wherein R_{A} is selected from the group consisting of hydrogen and C₁₋₅ alkyl; and R_{B} is selected from the group consisting of C₁₋₅ alkyl and —O—C₁₋₄ alkyl.

3. The compound or salt of claim 1 wherein R_{A} is hydrogen.

4. The compound or salt of claim 3 wherein R_{B} is C₁₋₅ alkyl.

5. The compound or salt of claim 1 wherein R_{A} and R_{B} are each methyl.

6. The compound or salt of claim 1 wherein —X— is 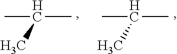
—CH₂—, —(CH₂)₂—, —CH(CH₃)—, —(CH₂)₃—, or —(CH₂)₄.

7. The compound or salt of claim 1 wherein R₁ is —X—R₄ wherein X is C₁₋₄ alkylene and R₄ is selected from the group consisting of aryl and heteroaryl.

8. The compound or salt of claim 7 wherein R₁ is benzyl, wherein the phenyl group is unsubstituted or substituted by one substituent selected from the group consisting of alkyl, alkoxy, haloalkyl, haloalkoxy, and halogen.

9. The compound or salt of claim 8 wherein R₁ is benzyl or 4-fluorobenzyl.

10. The compound or salt of claim 1 wherein R₁ is tetrahydro-2H-pyran-4-ylmethyl.

11. The compound or salt of claim 1 wherein R₁ is pyridin-3-ylmethyl, isoxazol-5-ylmethyl, or isoxazol-3-ylmethyl.

12. A pharmaceutical composition comprising a therapeutically effective amount of a compound or salt of claim 1 and a pharmaceutically acceptable carrier.

13. A method of inducing cytokine biosynthesis in an animal comprising administering an effective amount of a compound or salt of claim 1 to the animal.

14. A method of selectively inducing the biosynthesis of IFN-α in an animal comprising administering an effective amount of a compound or salt of claim 1 to the animal.

15. A method of treating a viral disease in an animal comprising administering a therapeutically effective amount of a compound or salt of claim 1 to the animal.

16. A method of treating a viral disease in an animal comprising administering a therapeutically effective amount of a compound or salt of claim 1 to the animal; and selectively inducing the biosynthesis of IFN-α in the animal.

17. A method of treating a neoplastic disease in an animal comprising administering a therapeutically effective amount of a compound or salt of claim 1 to the animal.

18. A method of treating a neoplastic disease in an animal comprising administering a therapeutically effective amount of a compound or salt of claim 1 to the animal; and selectively inducing the biosynthesis of IFN-α in the animal.
